(19) United States
(12) Patent Application Publication
(10) Pub. No.: US 2005/0277815 A1
(43) Pub. Date: Dec. 15, 2005

(54) Display method of test pattern and medical image display apparatus

(75) Inventors: Masataka Taniguchi, Tokyo (JP); Youichi Ono, Tokyo (JP)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: KONICA MINOLTA MEDICAL & GRAPHIC, INC.

(21) Appl. No.: 11/150,315

(22) Filed: Jun. 13, 2005

(30) Foreign Application Priority Data
Jun. 15, 2004 (JP) 2004-176983
Jun. 15, 2004 (JP) 2004-177008
Jun. 15, 2004 (JP) 2004-177073
Jun. 15, 2004 (JP) 2004-177104

(51) Int. Cl. A61B 5/00

(52) U.S. Cl. 600/300

(57) ABSTRACT

A display method of a test pattern makes a display unit of a medical image display apparatus display one of a plurality of test patterns for correcting a display gradation characteristic of the medical image display apparatus on one screen in a way of changing the test pattern every screen so that the test pattern displayed on each changed screen is displayed at a predetermined ratio to a size of a display screen and a luminance level of a background region of the test pattern is displayed at a different luminance level every change of the screen.

### BACKGROUND OF THE INVENTION

1. Field of the Invention

The present invention relates to a display method of a test pattern used at the time of correcting a display gradation characteristic of a medical image display apparatus based on a visual observation of an observer, and the medical image display apparatus displaying the test pattern.

2. Description of the Related Art

In the field of medicine, a monitor diagnosis of performing an interpretation of a radiogram by displaying a medical image of a patient, which has been obtained by various kinds of examination photographing such as X-ray radiographing, a magnetic resonance imaging (MRI) scan, and an ultrasonic wave scan, on a monitor has been prevalent. Moreover, it is also possible to perform the interpretation of the same image on monitors located at different places with the development of internal and external networks of a hospital. However, in the case where the monitors used for interpretations of radiograms are different kind ones such as a liquid crystal display (LCD) and a cathode ray tube (CRT), because their display characteristics differ from each other, even the same images would be differently seen sometimes. Moreover, even in the case of the same kind of monitors, sometimes the same images would also be seen differently from each other owing to the differences of their degrees of deterioration caused by their use, their installed environments, and the like.

Thus, when an image is differently seen on each monitor, the accuracy of diagnosis is thereby influenced. Consequently, it is necessary to unify the appearances of the images on the respective monitors by performing the correction of their display gradation characteristics (hereinafter referred to as calibration). Although the calibration is conventionally performed using a luminance meter, because the luminance meter is generally expensive, the calibration using the luminance meter brings about a high cost. Moreover, in the case where there are many monitors for interpretations of radiograms, the operation of performing the calibration of each monitor using the luminance meter one by one takes a lot of trouble, and very troublesome.

On the other hand, a method of confirming a calibration result by a visual observation of an operator after the implementation of the calibration using a luminance meter was proposed (see, for example, JP-Tokukaihei-11-327501A). The calibration method described in JP-Tokukaihei-11-327501A, as shown in FIG. 31, displays a plurality of test patterns displayed at luminance levels different from one another on a monitor to let an operator (observer) confirm the luminance level of each test pattern by visual observation.

However, because the adjoining test patterns come into the view of the operator together with a test pattern of a judgment object when the plurality of test patterns is displayed on one screen as shown in FIG. 31, the accuracy of the visibility of the operator falls, and it becomes difficult to perform accurate calibration.

Moreover, because the test patterns having the same shapes and the same sizes are always displayed at the same positions, an afterimage of the test pattern of the preceding judgment object may remain in the eyes of the operator. In this case, there is the possibility of the falling of the accuracy of the visibility of the operator owing to the influence of the preceding test pattern to the test pattern of the judgment object displayed later.

### SUMMARY OF THE INVENTION

It is an object of the present invention to improve the accuracy of the visibility of an operator to make it possible to perform the calibration by the visual observation of the operator.

For achieving the above object, in accordance with a first aspect of the present invention, a display method of a test pattern makes a display unit of a medical image display apparatus display one of a plurality of test patterns for correcting a display gradation characteristic of the medical image display apparatus on one screen in a way of changing the test pattern every screen so that the test pattern displayed on each changed screen is displayed at a predetermined ratio to a size of a display screen and a luminance level of a background region of the test pattern is displayed at a different luminance level every change of the screen.

According to the present invention, when the test patterns are displayed, one of the test patterns is displayed on one screen in the way of changing the test pattern every screen. Consequently, it is possible to prevent a fall of the accuracy of visibility caused by the entering of a test pattern other than the test pattern to be visually recognized into the visual field of an observer. Moreover, the size of the test pattern displayed on each changed screen is made to be a predetermined ratio to the size of a display screen, and the luminance level of the background region of the test pattern is made to be different one every change of the screen. Consequently, by varying the luminance level in the background region of a test pattern, the fall of the accuracy of the visibility can be prevented. Thus, more accurate calibration can be performed.

Moreover, it is preferable that the luminance level of the background region of the test pattern is made to correspond to the luminance level of the test pattern displayed on each changed screen.

According to the prevention, by varying the luminance level of the background region of the test pattern according to the luminance level of the test pattern, the eyes of an operator adapt themselves to the luminance level of the test pattern to be visually recognized, and the accuracy of the visibility is improved. Consequently, more accurate calibration can be performed.

Moreover, it is preferable that the test pattern displayed on each changed screen is displayed at a ratio of 10% of a size of the display screen.

According to the invention, the size of the test pattern is made to be 10% of the size of the display screen. The ratio of 10% is one regulated by the digital imaging and communication in medicine (DICOM) standard, and consequently it is possible to perform display in conformity with the standard.

Moreover, in accordance with a second aspect of the present invention, a display method of a test pattern makes a display unit of a medical image display apparatus display one of a plurality of test patterns for correcting a display gradation characteristic of the medical image display apparatus on one screen on the display section in a way of changing the test pattern every screen so as to vary a display form of the test pattern displayed on each changed screen.

According to the present invention, when the test patterns are displayed, one of the test patterns is displayed on one screen in the way of changing the test pattern every screen. Consequently, it is possible to prevent a fall of the accuracy of visibility caused by the entering of a test pattern other than the test pattern to be visually recognized into the visual field of an operator. Moreover, because the display form of the test pattern displayed on each changed screen is varied, it is possible to prevent an afterimage of the test pattern. Or, even when the afterimage of the test pattern remains, the influence of the afterimage to the test pattern to be visually recognized next can be prevented. Consequently, more accurate calibration can be performed.

It is preferable to vary the display form of the test pattern every change of the screen.

According to the invention, because the display form of the test pattern is varied every change of the screen, it is possible to prevent that the test pattern displayed on the preceding screen influences the visual recognition of the test pattern displayed on the succeeding screen.

It is preferable to vary the display form of the test pattern in each changed screen.

According to the invention, because the display form of the test pattern is varied in each changed screen, it is possible to prevent the generation of an afterimage of the test pattern by always varying the display form of the test pattern displayed in the one screen.

It is preferable that the display form is a display position of the test pattern.

According to the invention, in the case where the afterimage of the test pattern on the preceding screen remains when the display position of the test pattern is varied every change of the screen, the afterimage remains at a position different from the position of the test pattern to be visually recognized next. Consequently, it is possible to prevent the influence of the after image of the test pattern on the preceding screen to the test pattern to be displayed on the succeeding screen. On the other hand, when the display position is varied in each changed screen, the display position of the test pattern is not fixed, and consequently the generation of the afterimage thereof can be prevented.

It is preferable that the display form is a display size of the test pattern.

According to the invention, by varying the display size of the test pattern every change of the screen or in each changed screen, the display form of the test pattern is not fixed, and consequently the generation of the afterimage thereof can be prevented. Or, even when the afterimage has been generated, the influence thereof to the test pattern to be displayed on the succeeding screen can be decreased, and consequently a fall of the accuracy of the visibility of an operator can be prevented.

It is preferable that the display form is a display shape of the test pattern.

According to the invention, the display shape of the test pattern is varied. Consequently, by varying the display shape every change of the screen or in each changed screen, the display form of the test pattern is not fixed, and consequently the generation of the afterimage thereof can be prevented. Or, even when the afterimage has been generated, the influence thereof to the succeeding test pattern can be decreased, and consequently a fall of the accuracy of the visibility of an operator can be prevented.

Moreover, in accordance with a third aspect of the present invention, a display method of a test pattern makes a display unit of a medical image display apparatus display one of a plurality of test patterns for correcting a display gradation characteristic of the medical image display apparatus on one screen on the display unit in a way of changing the test pattern every screen so that the test pattern displayed on each changed screen comprises two display regions to display one display region having a larger display area between them at a lower luminance level than that of another display region.

According to the present invention, when the test patterns are displayed, one of the test patterns is displayed on one screen in the way of changing the test pattern every screen. Consequently, it is possible to prevent a fall of the accuracy of visibility caused by the entering of a test pattern other than the test pattern to be visually recognized into the visual field of an observer. Moreover, because one display region having a larger display area between the two display regions constituting the test pattern is displayed at a lower luminance level, the lower luminance region is wholly larger when the observer observes the test pattern. Consequently, the pupils of the observer easily open, and become sensitive to light. Thus, the accuracy of visibility to luminance levels is improved. Consequently, the correction of the display gradation characteristic can be performed more accurately.

Moreover, it is preferable that the test pattern displayed on each changed screen is displayed on the whole screen.

According to the invention, because the test pattern is displayed on the whole screen, the occupation rate of the display region of a low luminance level in the visual field of an observer becomes larger, and the accuracy of visibility of the observer can be improved.

In accordance with a fourth aspect of the present invention, a display method of a test pattern makes a display unit of a medical image display apparatus display one of a plurality of test patterns for correcting a display gradation characteristic of the medical image display apparatus on one screen in a way of changing the test pattern every screen so that a display condition of the test pattern displayed on the screen is determined based on preset information of a visual characteristic and an observation condition of an observer to display the test pattern under the determined display condition.

According to the present invention, when the test patterns are displayed, one of the test patterns is displayed on one screen in the way of changing the test pattern every screen. Consequently, it is possible to prevent a fall of the accuracy of visibility caused by the entering of a test pattern other than the test pattern to be visually recognized into the visual field of the observer of the test pattern. Moreover, because the test pattern is displayed under the display condition based on the visual characteristic and the observation condition of the observer, the visual recognition rate of the observer to the test pattern is improved, and the correction of the display gradation characteristic can be performed more accurately.

Moreover, it is preferable that a background region of the test pattern displayed on each changed screen is displayed at a fixed luminance level.

According to the invention, because the luminance level of the background region of the test pattern is unified at the fixed luminance level on each changed screen, it is possible to decrease the influence on the visual recognition force of the observer by the luminance level of the background region to that of the test pattern displayed on each changed screen, and a stable correction of the display gradation characteristic can be performed.

It is preferable that a background region of the test pattern displayed on each changed screen is made to be displayed at a luminance level corresponding to a luminance level of the test pattern on each changed screen.

According to the invention, because the luminance level of the background region of the test pattern displayed on each changed screen varies according to the luminance level when the test pattern is displayed, the background region having the same or about the same luminance level as the luminance level of the test pattern enters the visual field of the observer together with the test pattern. Thereby, the observer adapts himself or herself to the luminance level, and the accuracy of visibility is improved. Consequently, more accurate correction of the display gradation characteristic can be performed.

It is preferable that each of the test patterns comprises a plurality of line pairs, each of the line pairs comprising two line-like display regions to be displayed at luminance levels different from each other, and that, when each of the test patterns is displayed on each changed screen, a density of the line pairs formed in the test pattern is determined based on information of a visual characteristic and an observation condition of an observer as a display condition of the test pattern to display the test pattern at the determined line pair density.

According to the invention, by determining the density of the line pairs formed in the test pattern in order that the visual recognition rate of the observer may be improved, based on the information of the visual characteristic and the observation condition of the observer as the display condition, the correction of the display gradation characteristic can be performed more accurately.

It is preferable that the observation condition is an observation distance from the observer to a display screen and a pattern size of the test pattern.

According to the invention, the display condition of the test pattern for improving the visual recognition rate of the observer can be determined based on the observation condition comprising the observation distance and the pattern size of the test pattern.

Moreover, in accordance with a fifth aspect of the present invention, a medical image display apparatus is equipped with a display unit for displaying a medical image, and a control unit for making the display unit display one of a plurality of test patterns for correcting a display gradation characteristic of the medical image display apparatus on one screen in a way of changing the test pattern every screen so that the test pattern displayed on each changed screen is displayed at a predetermined ratio to a size of a display screen and a luminance level of a background region of the test pattern is displayed at a different luminance level every change of the screen.

According to the present invention, when the test patterns are displayed, one of the test patterns is displayed on one screen in the way of changing the test pattern every screen. Consequently, it is possible to prevent a fall of the accuracy of visibility caused by the entering of a test pattern other than the test pattern to be visually recognized into the visual field of an observer. Moreover, the size of the test pattern displayed on each changed screen is made to be a predetermined ratio to the size of a display screen, and the luminance level of the background region of the test pattern is made to be different one every change of the screen. Consequently, by varying the luminance level in the background region of a test pattern, the fall of the accuracy of the visibility can be prevented. Thus, more accurate calibration can be performed.

Moreover, in accordance with a sixth aspect of the present invention, a medical image display apparatus is equipped with a display unit for displaying a medical image, and a control unit for making the display unit display one of a plurality of test patterns for correcting a display gradation characteristic of the medical image display apparatus on one screen in a way of changing the test pattern every screen so as to vary a display form of the test pattern displayed on each changed screen.

According to the present invention, when the test patterns are displayed, one of the test patterns is displayed on one screen in the way of changing the test pattern every screen. Consequently, it is possible to prevent a fall of the accuracy of visibility caused by the entering of a test pattern other than the test pattern to be visually recognized into the visual field of an operator. Moreover, because the display form of the test pattern displayed on each changed screen is varied, it is possible to prevent an afterimage of the test pattern. Or, even when the afterimage of the test pattern remains, the influence of the afterimage to the test pattern to be visually recognized next can be prevented. Consequently, more accurate calibration can be performed.

In accordance with a seventh aspect of the present invention, a medical image display apparatus is equipped with a display unit for displaying a medical image, and a control unit for making the display unit display one of a plurality of test patterns for correcting a display gradation characteristic of the medical image display apparatus on one screen in a way of changing the test pattern every screen so that the test pattern displayed on each changed screen comprises two display regions to display one display region having a larger display area between them at a lower luminance level than that of another display region.

According to the present invention, when the test patterns are displayed, one of the test patterns is displayed on one screen in the way of changing the test pattern every screen. Consequently, it is possible to prevent a fall of the accuracy of visibility caused by the entering of a test pattern other than the test pattern to be visually recognized into the visual field of an observer. Moreover, because one display region having a larger display area between the two display regions constituting the test pattern is displayed at a lower luminance level, the lower luminance region is wholly larger when the observer observes the test pattern. Consequently, the pupils of the observer easily open, and become sensitive to light. Thus, the accuracy of visibility to luminance levels is improved. Consequently, the correction of the display gradation characteristic can be performed more accurately.

In accordance with an eighth aspect of the present invention, a medical image display apparatus is equipped with a display unit for displaying a medical image, and a control unit for making the display unit display one of a plurality of test patterns for correcting a display gradation characteristic in the display unit on one screen in a way of changing the test pattern every screen so that a display condition of the test pattern displayed on the screen is determined based on preset information of a visual characteristic and an observation condition of an observer to display the test pattern under the determined display condition.

According to the present invention, when the test patterns are displayed, one of the test patterns is displayed on one screen in the way of changing the test pattern every screen. Consequently, it is possible to prevent a fall of the accuracy of visibility caused by the entering of a test pattern other than the test pattern to be visually recognized into the visual field of the observer of the test pattern. Moreover, because the test pattern is displayed under the display condition based on the visual characteristic and the observation condition of the observer, the visual recognition rate of the observer to the test pattern is improved, and the correction of the display gradation characteristic can be performed more accurately.

### BRIEF DESCRIPTION OF THE DRAWINGS

The present invention will be fully understood from the following detailed description taken in conjunction with the accompanying drawings, but those should not be interpreted to restrict the present invention to them, in which:

FIG. 1 is a view showing the inner configuration of a medical image display apparatus in the present embodiment;

FIG. 2 is a view showing test patterns displayed at the time of calibration processing;

FIG. 3 is a view illustrating two display regions constituting a test pattern;

FIG. 4 is a view showing examples of test patterns comprising line pairs;

FIG. 5 is a view illustrating line pairs constituting a test pattern;

FIG. 6 is a flowchart illustrating the calibration processing executed by the medical image display apparatus;

FIG. 7 is a view showing examples of display screens of test patterns (rectangular patterns);

FIG. 8 is a view showing examples of display screens of test patterns (line patterns);

FIG. 9 is a view showing a function g(DDL);

FIG. 10 is a view showing a function h(DDL);

FIG. 11 is a view showing a correction curve for correcting a display gradation characteristic of the medical image display apparatus;

FIG. 12 is a graph showing the accuracy of the visibility of a person in cases of variation adaptation and fixation adaptation;

FIG. 13 is a flowchart illustrating a first pattern display processing executed by the medical image display apparatus;

FIG. 14 is a view showing examples of display screens of test patterns;

FIG. 15 is a flowchart illustrating LUT creation processing executed by the medical image display apparatus;

FIG. 16 is a view showing examples of displays of test patterns comprising line pairs;

FIG. 17 is a flowchart illustrating a second pattern display processing;

FIG. 18 is a view showing examples of display screens of test patterns;

FIG. 19 is a view showing examples of display screens of test patterns in the case of line patterns;

FIG. 20 is a flowchart illustrating a third pattern display processing;

FIG. 21 is a view showing display examples of test patterns;

FIG. 22 is a view showing examples of display screens in the case where the shapes of test patterns are varied;

FIG. 23 is a flowchart illustrating the calibration processing executed by the medical image display apparatus;

FIG. 24A is a view showing an example of a test pattern displayed on the whole display screen in the state of being adjusted in size so that the display area of a reference target is larger than that of an adjustment target;

FIG. 24B is a view showing an example of a test pattern displayed on the whole display screen in the state of being adjusted in size so that the display area of the reference target is larger than that of the adjustment target;

FIG. 25 is a view showing screen transitions when a test pattern is sequentially displayed on a screen;

FIG. 26 is a flowchart illustrating the calibration processing executed by the medical image display apparatus;

FIG. 27 is a view showing a visual field range in case of a predetermined visual field angle and a predetermined observation distance;

FIG. 28A is view showing an example of a test pattern and a display drive of the background region thereof at a fixed drive level;

FIG. 28B is a view showing another example of a test pattern and a display drive of the background region thereof at a fixed drive level;

FIG. 29 is a view showing an example of a display of a test pattern in which lines are arranged so as to be horizontal lines;

FIG. 30 is a view showing examples of display drives of background regions of test patterns at the same drive levels as those of the reference targets of the test patterns; and

FIG. 31 is a view showing examples of conventional test patterns.

### DETAILED DESCRIPTION OF THE PREFERRED EMBODIMENTS

Hereinafter, embodiments pertaining to a display method of a test pattern and a medical display image apparatus of the present invention are described with reference to the attached drawings.

### First Embodiment

First the configuration thereof is described.

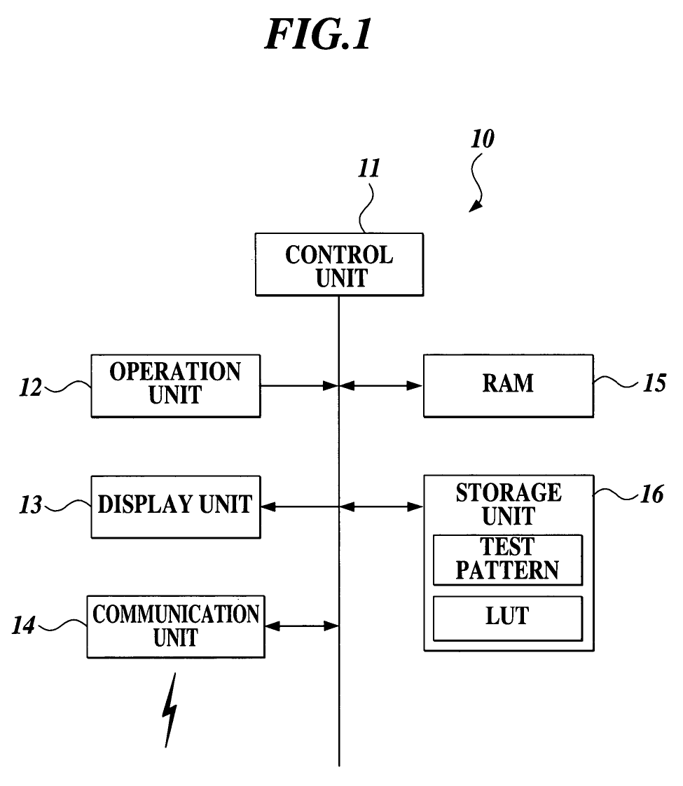

The internal configuration of a medical image display apparatus 10 in a first embodiment is shown in FIG. 1.

As shown in FIG. 1, the medical image display apparatus 10 comprises a control unit 11, an operation unit 12, a display unit 13, a communication unit 14, a random access memory (RAM) 15, and a storage unit 16.

The control unit 11 unwinds various control programs, which are stored in the storage unit 16, such as a system program and a calibration processing program according to the present invention, to the RAM 15, and performs the centralized control of the operation of each unit in conformity with the control program.

In the calibration processing, the level range of drive levels at which the medical image display apparatus 10 can perform display drives is divided into N equal parts, and the data of each of N test patterns in which the drive levels obtained by the division into N equal parts are set severally is outputted to the display unit 13 together with the set drive level information to be displayed on the display unit 13. That is, each test pattern is displayed at the luminance level according to each drive level obtained by the division into N equal units. Each test pattern comprises two display regions. In one region of them, the drive level obtained by the division into N equal parts is set, and in the other region, the drive level thereof is set to be higher than the drive level obtained by the division into the N equal parts by a predetermined level.

When the N test patterns are displayed on the display unit 13, the control unit 11 generates screen data of locating one test pattern on one screen, and outputs the generated screen data to the display unit 13 together with the information of the drive level set to the test pattern. Thereby, the control unit 11 displays one test pattern on one screen in the way of changing the test pattern every screen. Moreover, at the time of generating the screen data, the control unit 11 expands or reduces the test pattern to perform a size alteration so that the size of the test pattern displayed in each changed screen may be at a ratio of 10% of the size of the display screen, and sets the drive level of the background region of the test pattern so that the background region may be displayed at a luminance level corresponding to the test pattern. Incidentally, the ratio of 10% is a ratio prescribed in the DICOM standard. That is, a control section can be realized by the cooperation of the control unit 11 and the calibration processing program.

Then, when an adjustment operation of the luminance level of the displayed test pattern has been performed through the operation unit 12, the control unit 11 alters the drive level of the test pattern according to the operation instruction to make the display unit 13 perform a display drive. When the adjustment operation has ended, the control unit 11 obtains a correction curve for correcting the display gradation characteristic of the medical image display apparatus 10 based on the value of the drive level of each line constituting each test pattern. Then, the control unit 11 creates a look up table (LUT) in which output values to input values of the correction curve are prescribed, and stores the LUT in the storage unit 16.

Incidentally, the display characteristic means correlations between drive levels and luminance levels, and the display gradation characteristic means correlations between input levels of pixel values in image data of a display object and the luminance levels at the time of displaying the image data.

The operation unit 12 includes a keyboard and a mouse. When the keyboard or the mouse is operated, the operation unit 12 generates an operation signal according to the operation, and outputs it to the control unit 11. Incidentally, the operation unit 12 may include a touch panel which is integrally formed with the display unit 13, and the like.

The display unit 13 has a monitor such as an LCD and a CRT, and drives the monitor in conformity with a display control signal inputted from the control unit 11. The display unit 13 displays one of the test patterns [n] inputted from the control unit 11 on one screen in the way of changing the test pattern [n] every screen at the time of calibration processing. Incidentally, when the display unit 13 displays each test pattern [n], the display unit 13 performs the display drive of the monitor at the specified drive level in conformity to the drive level information inputted together with the test pattern [n].

The communication unit 14 includes communication interfaces such as a network interface card (NIC), a modem, and a router, and performs data communication with external apparatuses on a local area network (LAN) provided in a hospital through the communication interfaces. For example, the communication unit 14 accesses an image server managing medical images of patients made to be a database to obtain the data of a medical image.

The RAM 15 forms a work area which temporarily stores various programs to be executed by the control unit 11, the data processed by these programs, and the like.

The storage unit 16 comprises a magnetic or an optical recording medium, a semiconductor memory, or the like, and stores various control programs such as a system program and the calibration processing program. Moreover, the storage unit 16 stores the information of the ratio (10% in the present embodiment) of the display size of a test pattern to the size of the display screen, and the information of the size of the display screen of the display unit 13 and the like as parameters used at the time of the calibration processing. The storage unit 16 further stores, for example, a LUT for correcting the display gradation characteristic of the medical image display apparatus 10 created by the calibration processing as a processing result of a program.

Moreover, the storage unit 16 stores the data of a plurality of test patterns used in the calibration processing.

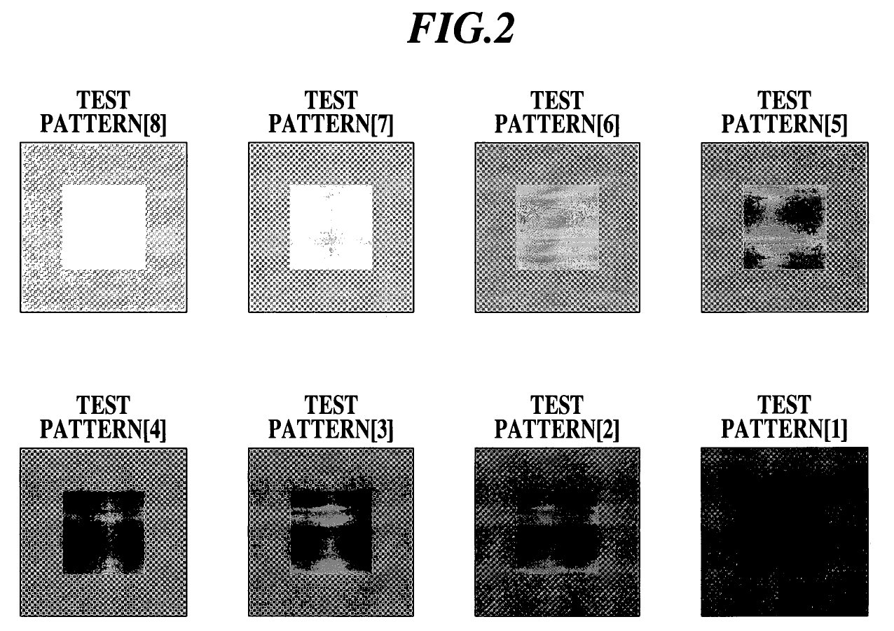

Examples of test patterns are shown in FIG. 2.

As shown in FIG. 2, the test patterns [n] (n in the bracket denotes a pattern number given for identifying each test pattern. n=1, . . . , N) are created according to respective drive levels which are divided parts of the division of the level range of the drive levels capable of being driven by the display unit 13, namely a range of from 0 to the maximum drive level DDLₘₐₓ, into N equal parts. Incidentally, although an example of division into eight equal parts is shown in the present embodiment, the number of N of the equal division is made to be suitably set according to the accuracy of calibration. In the case where the calibration is performed at high accuracy, it is preferable to increase the number of N.

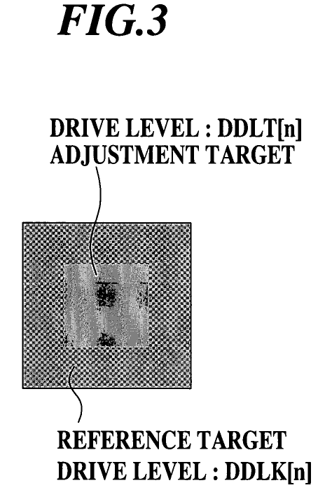

In each test pattern [n], two display regions in which different drive levels are set are formed as shown in FIG. 3. One of the drive levels of the division of the maximum drive level DDLₘₐₓ into N equal parts is set in one display region, and a drive level higher than the drive level set in the one display region by a fixed level is set in the other display region. The display region where the drive level of one of the divided N equal parts is set is referred to as a reference target, and the display region where the drive level so that the display region is at a higher luminance level than that of the reference target is referred to as an adjustment target.

When the drive level of the reference target is denoted by DDLK [n] and the drive level of the adjustment target is denoted by DDLT [n], DDLK [n] is expressed by the following formula 1, and DDLT [n] is expressed by the following formula 2:

$$DDLK [ n ] = \frac{n - 1}{N - 1} \times {DDL}_{max} \qquad formula 1$$

DDLT[n]=DDLK[n]+ΔDDLᵢₙᵢₜ formula 2
where ΔDDLᵢₙᵢₜ denotes a constant.

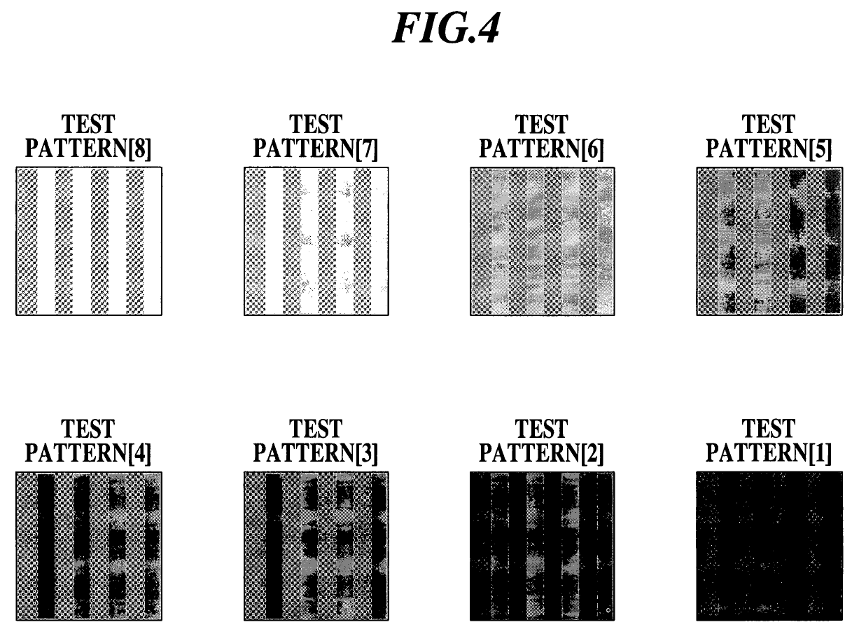

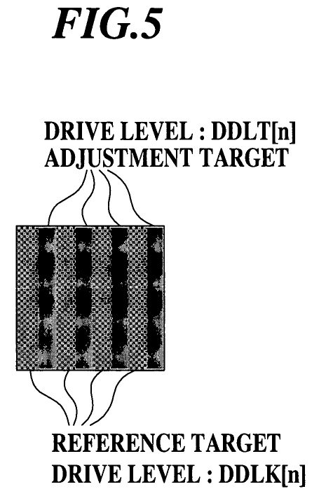

Incidentally, the test patterns [n] are not limited to those formed of the display regions shaped in squares as shown in FIGS. 2 and 3, but the test patterns [n] formed of the display regions shaped in lines as shown in FIGS. 4 and 5. Also in this case, the maximum drive level DDLₘₐₓ is similarly divided into N equal parts, and N test patterns [n] in which each of the divided N equal drive levels is set are created.

In each test pattern [n] shown in FIG. 4, a plurality of line pairs are formed, in which each line pair comprises two line-like display regions in which different drive levels from each other are set, as shown in FIG. 5. In one line of each line pair, one of the drive levels obtained by the division into N equal parts of the maximum drive level DDLₘₐₓ is set, and in the other line, the drive level higher than the drive level set in the one line by a fixed level is set. The line in which one of the drive levels obtained by the division into N equal parts is set is the reference target, and the line in which the drive level is set in order that the line may be displayed at a luminance level higher than that of the reference target is the adjustment target.

Incidentally, in the following description, in order to distinguish the kinds of the test patterns [n], the test patterns which are shown in FIGS. 2 and 3 may be referred as rectangular patterns, and the test patterns shown in FIGS. 4 and 5 may be referred to as line patterns.

Next, the calibration processing executed by the medical image display apparatus 10 of the present embodiment is described using the test patterns [n].

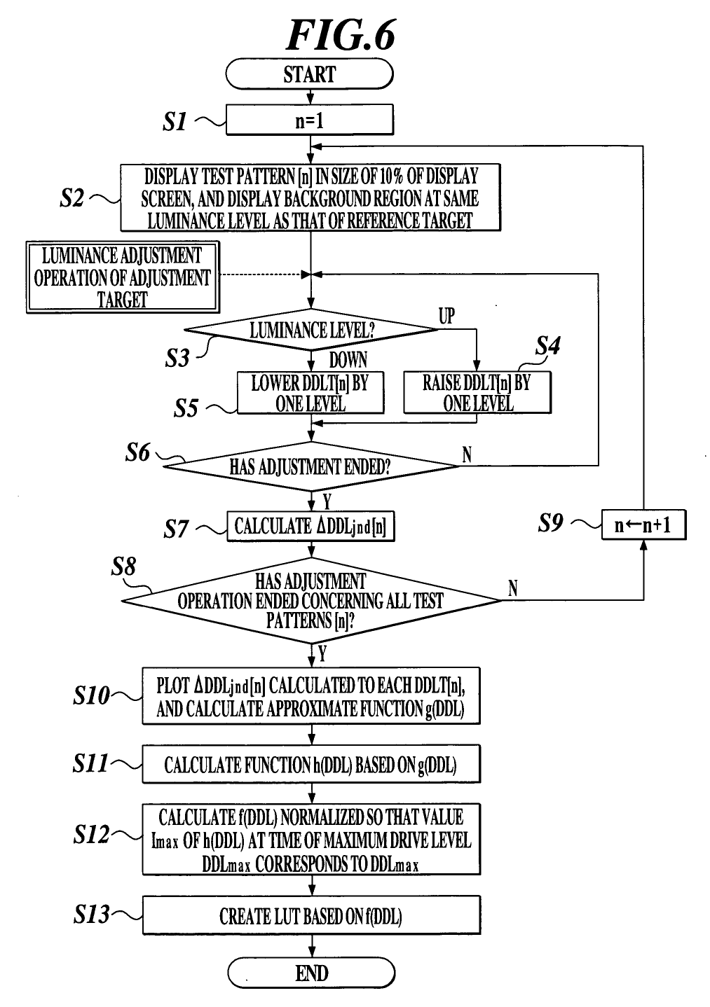

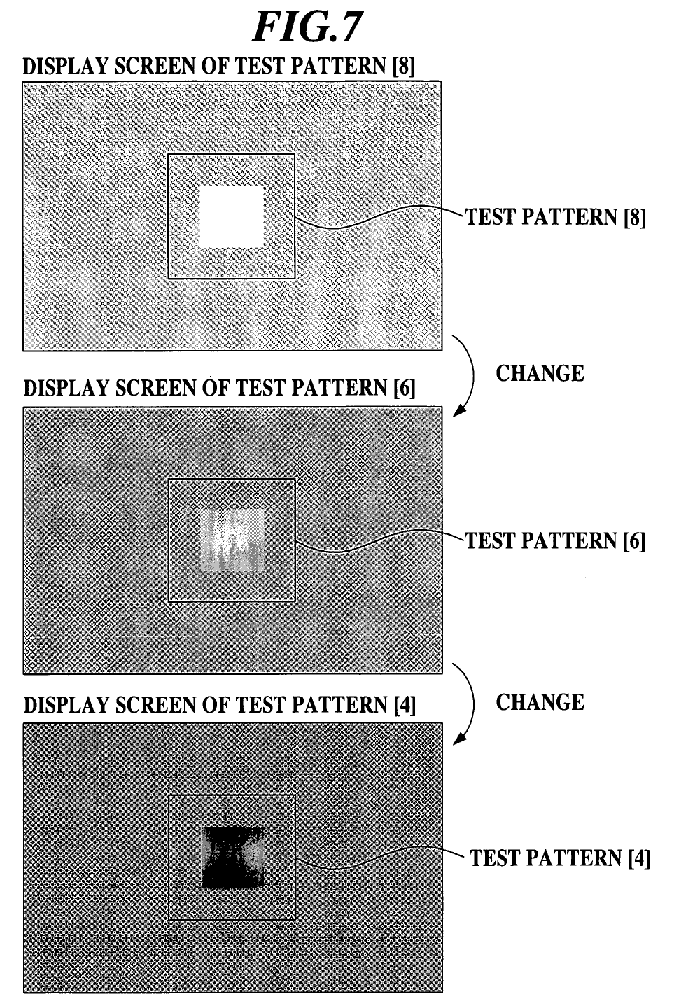

FIG. 6 is a flowchart illustrating the calibration processing, and FIG. 7 is a view showing a transition diagram of the display screens displayed at the time of the processing. The processing is started when the calibration is selected in an environment setting menu.

In the calibration processing shown in FIG. 6, the pattern number n of a test pattern to be displayed is first set to n=1, which is an initial value (Step S1). When the pattern number n has been set, in the control unit 11, the data of the test pattern [n] having the pattern number n is read from the storage unit 16, and expansion or reduction is performed in order that the size of the test pattern [n] may be 10% of the size of the display screen. Then, display screen data is generated. The display screen data indicates the location of the test pattern [n] in the altered size by the expansion or the reduction at the central part on the display screen and the setting of the drive level of the background region of the test pattern [n] to be the same level as the drive level DDLK [n] of the reference target. The generated display screen data is outputted to the display unit 13 together with the drive level information of each of the display regions. In the display unit 13, the display drive of the monitor is performed in conformity with the drive level information, and the display screen including the test pattern [n] is displayed (Step S2).

FIG. 7 shows examples of the display screens of test patterns [8], [6], and [4]. When the case where the test pattern [6] is displayed is described as an example, because only one pattern is displayed on one screen and the background region thereof is driven to be displayed by the same drive level as that of the reference target, the background region is displayed at the same luminance level as that of the display region of the reference target. Moreover, the test pattern [6] is displayed at the ratio of 10% of the size of the display screen. Although the ratio is one in accordance with the DICOM standard, it may be adopted to experimentally obtain and set the ratio at which the accuracy of the visibility is improved based on the visual characteristic of an operator (observer) in the relation of the size of the display screen without limiting the ratio to the 10%.

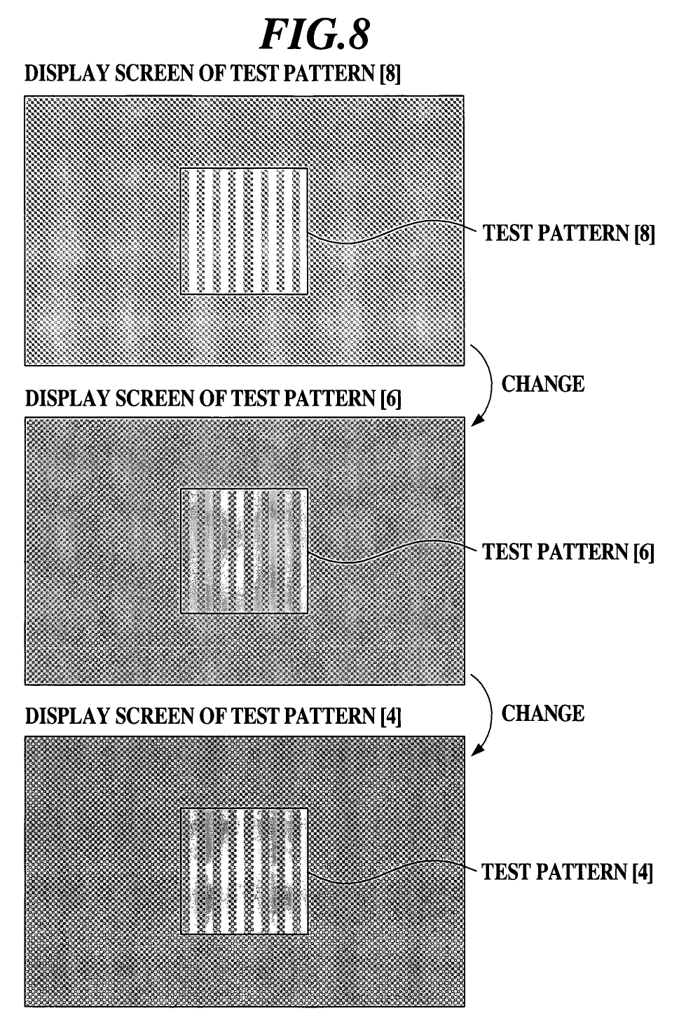

Incidentally, although the case where the test pattern [n] is a rectangular pattern is shown in FIG. 7, it may be adopted that a line pattern is applied as shown in FIG. 8. In the case of the line pattern, the line pattern is similarly displayed in the size of 10% of the display screen, and the background region of the line pattern is driven to be displayed at the same drive level as the drive level DDLK [n] of the reference target. Then, the background region and the reference target are displayed at the same luminance level.

Moreover, although the test pattern [n] is located at the central part of the display screen in FIGS. 7 and 8, the location position of the test pattern [n] is not especially limited. Because the degree of the influence of circumference light such as indoor lighting and sunlight differs according to a display position in case of observing a medical image, in spite of being displayed at the same luminance level, the luminance levels which the operator visually recognizes may differ depending on positions. Consequently, when the position at which a medical image is displayed is fixed at a predetermined position in a screen, the calibration in consideration of the environmental conditions at the time of observing the medical image can be performed by locating the test pattern [n] at a position corresponding to the display position of the medical image to let the operator judge the test pattern [n].

Moreover, depending on a region at which interpretation of radiogram is performed, the concern of an interpretation physician may concentrate into a specific region. For example, when interpretation of radiogram of a medical image in which a breast is photographed is performed, the concern is especially concentrated to the region of a mammary gland portion. In order to adjust the display gradation characteristic according to such an interested region, the regions where the interested region on a display screen is frequently displayed are experimentally obtained beforehand. When the test pattern [n] is displayed, the test pattern [n] is located in such an interested region, and the luminance level of the other region is displayed in accordance with that of the reference target. Thereby, effective calibration regarding the interested region as an important one can be performed. Incidentally, in this case, the display positions of the test patterns [n] are located at the substantially same positions in the interested regions over the respective changed screens.

When a test pattern [n] has been displayed in such a way, the operator performs an adjustment operation of raising or lowering the luminance level of an adjustment target until the luminance level difference between the adjustment target and the reference target in the test pattern [n] becomes the minimum luminance level difference which can be visually recognized by visual observation through the operation unit 12. The minimum luminance level difference which can be visually recognized means a luminance level difference at the limit of visually recognizing the luminance level difference as a result of an operator's adjusting the luminance level of the adjustment target in accordance with the luminance level of the reference target. To put it concretely, the luminance level is adjusted until a stage at which the operator would be unable to identify the luminance level difference between the adjustment target and the reference target if the luminance level of the adjustment target were lowered by more one level.

In the medical image display apparatus 10, when an operation of luminance adjustment of an adjustment target is made through the operation unit 12, the signal analysis of the operation signal is performed, and it is distinguished which the operation instruction is directed to raising the luminous level or to lowering the luminous level (Step S3). When the operation instruction is directed to raising the luminance level (Step S3; up), the drive level DDLT [n] of the adjustment target is increased by one level, and the adjustment target during being displayed is driven to be displayed at the increased drive level DDLT (Step S4). On the other hand, when the operation instruction is performed in order to lower the luminance (Step S3; down), the drive level DDLT [n] is lowered by one level, and the adjustment target during being displayed is driven to be displayed at the lowered drive level DDLT [n] (Step S5). That is, in response to one operation, a display is performed at a luminance level raised or lowered by one drive level. Incidentally, even when the operation of lowering the luminance level of the adjustment target is repeated, the adjustment operation is made to be restricted lest the luminance level of the adjustment target should be equal to the luminance level of the reference target or less by invalidating the adjustment operation of making the luminance level equal to the drive level of the reference target or less, or by a similar measure.

When the adjustment operation of the drive level has been performed, it is judged whether the adjustment operation has ended or not (Step S6). When the adjustment operation is still being performed through the operation unit 12 (Step S6; N), the processing returns to the process of Step S3, and the luminance level is adjusted in conformity with the inputted operation instruction. On the other hand, when the adjustment operation has ended (step S6; Y), a drive level DDLT′ [n] of the adjustment target of the test pattern [n] at the time of the adjustment end is judged, and a difference ΔDDL_{jnd} [n] between the drive level DDLT′ [n] and the drive level DDLK [n] of the reference target is calculated (Step S7). The difference ΔDDL_{jnd} [n] corresponds to the minimum luminance level difference which the operator can visually recognize in the test pattern [n].

Subsequently, it is judged whether the adjustment operation mentioned above has ended about all test patterns [n] or not (Step S8). When the adjustment operation has not ended about all of the test patterns [n] (step S8; N), the pattern number n is incremented by only one (Step S9), and the processing returns to the process of Step S2. That is, the display screen of the next test pattern [n] is displayed, and the adjustment operation is repeated.

As described above, by advancing the pattern number n and repeating the processes from Steps S2 to S7, in the display unit 13, as shown in FIGS. 7 and 8, only one test pattern [n] is displayed on one screen, and each test pattern [n] is sequentially changed and displayed in the order of the pattern number. Moreover, whenever the screen is changed, the luminance level of the background region of the test pattern [n] on each changed screen is displayed at the same luminance level as that of the reference target of the test pattern [n] displayed on the screen. That is, the background region is displayed at a different luminance level in accordance with the test pattern [n] displayed on each changed screen whenever the screen is changed.

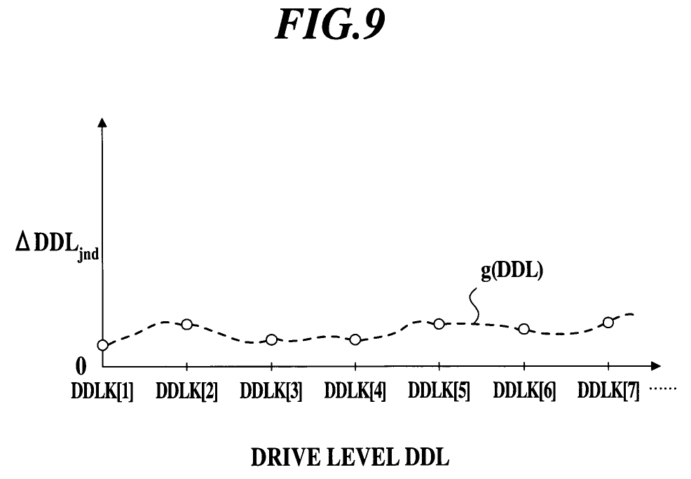

Then, when all the test patterns [n] have been displayed and the adjustment operation has ended (step S8; Y), as shown in FIG. 9, the difference ΔDDL_{jnd} [n] calculated to the drive level DDLK [n] of the reference target of each test pattern [n] is plotted, and an approximate function g(DDL) of each plotted point is calculated (Step S10). That is, each difference ΔDDL_{jnd} [n], which is a discrete value, is interpolated, and a continuous value corresponding to the drive level DDL of all level ranges is obtained. That is, the approximate function g(DDL) is a function showing a correspondence relation between the drive level DDL and the minimum drive level difference ΔDDL_{jnd} [n].

Subsequently, a function h(DDL) of the integral of the calculated function g(DDL) to every drive level DDL is calculated by the following formula 3 (Step S11). The above function g(DDL) expresses the inclination of the function h(DDL).

$$h ( DDL ) = {\int }_{0}^{DDL} g ( DDL ) ⅆ DDL \qquad formula 3$$

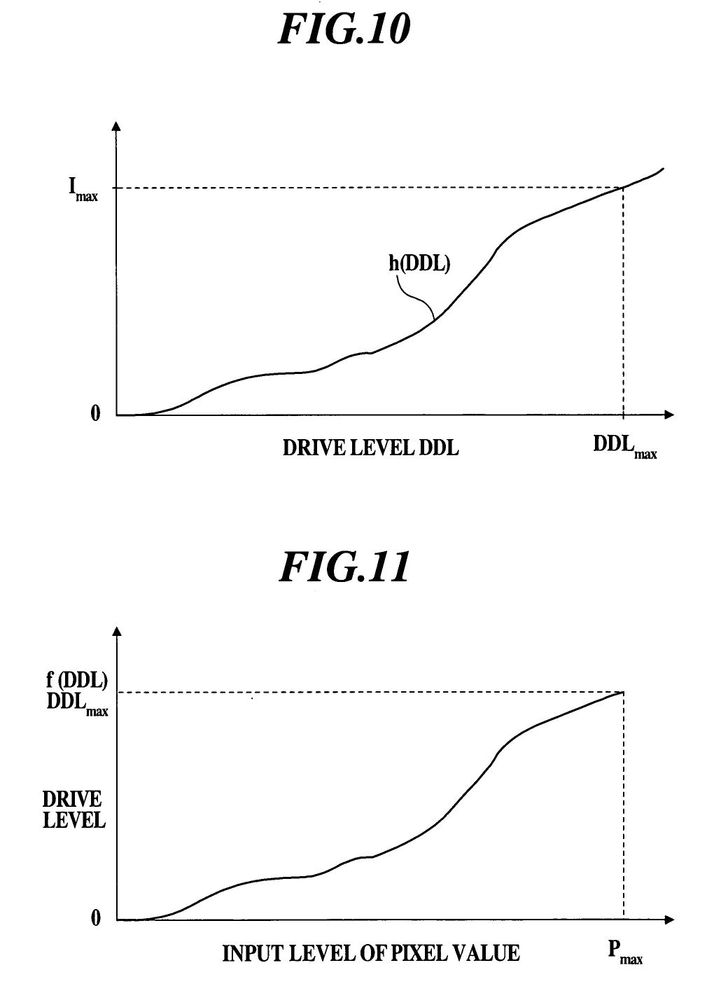

FIG. 10 is a view showing the function h(DDL). The X-axis thereof expresses the drive level DDL, and the Y-axis thereof expresses the function h(DDL) to the drive level DDL. In this case, supposing that a value of the function h(DDL) corresponding to the maximum drive level DDLₘₐₓ which can be driven by the medical image display apparatus 10 is set to Iₘₐₓ, the function h(DDL) (i.e. the values of the Y-axis) is normalized so that the value Iₘₐₓ corresponds to the maximum drive level DDLₘₐₓ, and a function f(DDL) expressing the normalized function h(DDL) is calculated by the following formula 4 (Step S12).

$$f ( DDL ) = \frac{h ( DDL )}{{I}_{max}} \times {DDL}_{max} \qquad formula 4$$

Then, a curve shown in FIG. 11 is created. The curve is obtained by substituting the drive levels DDL, which are the units of the X-axis of FIG. 10, with the input levels of image values (Pₘₐₓ in the figure indicates the maximum gradation which can be expressed by the medical image display apparatus 10), and by substituting the function h(DDL), which is the unit of the Y-axis, with the calculated function f(DDL). With this curve, a drive level which realizes a corresponding luminance level from the input level of a pixel value can be obtained so that the input level of the pixel value may be finally displayed at a luminance level visually linear to the input level of the pixel value. That is, the curve is a correction curve for correcting the display gradation characteristic according to the display characteristic of the medical image display apparatus 10 (that is, for correcting a relation between the input value of a pixel value and a luminance level at the time of being actually displayed according to the input level). In the control unit 11, the output values to the input values of this correction curve are calculated and are tabled to create a LUT (Step S13). When the created LUT is stored in the storage unit 16 as the LUT for correcting the display gradation characteristic of the medical image display apparatus 10, the present processing ends.

When a medical image is displayed, the created LUT is referred to, and the drive level (output value) corresponding to the input level (input value) of the pixel value of the medical image is obtained to perform a display and a drive at the drive level. Because the drive level obtained from the LUT is a drive level corrected in order that the luminance level at the time of being driven to be displayed may be in a visually linear relation with the input level of the pixel value, the drive and the display mean that the display gradation characteristic of the medical image display apparatus 10 is corrected according to the display characteristic of the medical image display apparatus 10 and the visual characteristic of the operator.

As mentioned above, according to the present embodiment, when the test patterns for calibration are displayed, the test patterns are changed to be displayed so that one pattern is displayed on one screen. Consequently, a fall of the accuracy of the visibility owing to the entering of a test pattern other than the test pattern to be visually recognized into the visual field of an operator can be prevented. Consequently, more accurate calibration can be performed.

Moreover, because the background region of the test pattern [n] displayed on each changed screen is displayed at the same luminance level as that of the reference target of the test pattern [n], the background region of the same luminance level as that of the test pattern [n] enters the visual field of the operator together with the test pattern [n]. Consequently, the eyes of the operator adapt themselves to the luminance level, and the accuracy of the visibility improves.

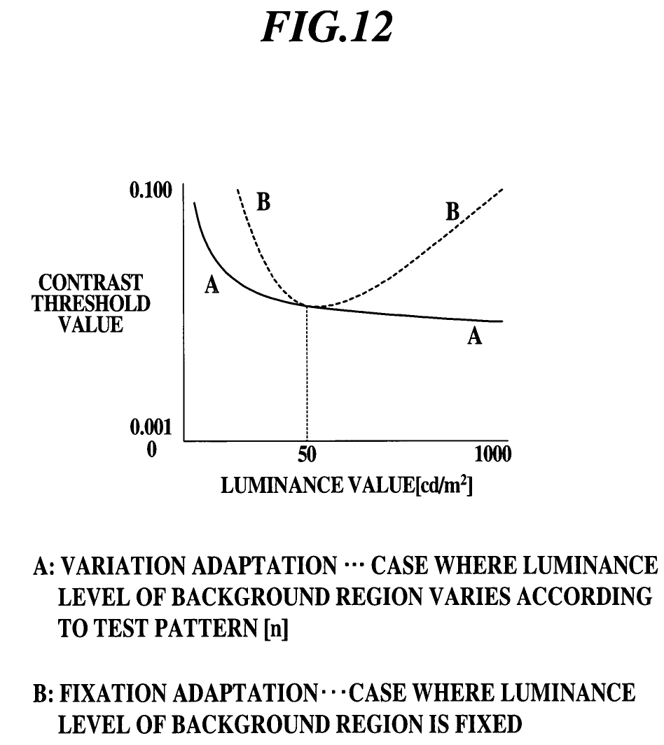

In a graph shown in FIG. 12, contrast threshold values (Y-axis) obtained by dividing the minimum luminance level differences ΔDDL_{jnd} [n] which the operator visually recognizes to each luminance value (X-axis; unit: cd/m²) when the test patterns [n] are actually displayed by the average luminance of reference targets and adjustment targets are plotted. That is, the graph shows that as the value of the contrast threshold value is smaller, the accuracy of the visibility is more improved.

In the graph, a solid line A expresses measurement results in the case (hereinafter the case is referred to as variation adaptation) where the judgments of the test patterns [n] are performed on the display screens on which the luminance levels of the background regions are made to correspond to the luminance levels of the test patterns [n] according to the present invention. A dotted line B expresses measurement results in the case (hereinafter the case is referred to as fixation adaptation) where the judgments of the test patterns [n] are performed on the display screens on which the luminance levels of the background regions are fixed to a certain luminance level (the luminance value at this time is 50 cd/m²) independent of the luminance levels of the test patterns [n].

As shown in FIG. 12, in the region in which the luminance values are 50 cd/m² or less, the contrast threshold values of the case of the variation adaptation indicated by the solid line A are smaller than those of the case of the fixation adaptation indicated by the dotted line B, and it is known that the accuracy of the visibility in the case of the variation adaptation is more improved than that in the case of the fixation adaptation.

Incidentally, although the luminance levels of the background regions are made to be the same luminance levels as those of the reference targets of the test patterns [n] in the present embodiment, as long as the luminance levels of the background regions vary according to the luminance levels of the test patterns [n], the drive levels of the background regions may be set to be other values such as average values of the drive levels DDLK [n] of the reference targets and the drive levels DDLT [n] of the adjustment targets or a set value in the range of from the drive levels DDLK [n] to the drive levels DDLT [n].

Furthermore, because the test patterns [n] are displayed after their size alterations have been performed so that the size of the test pattern [n] displayed on each changed screen is that of a predetermined ratio to the size of the display screen, the test patterns [n] can be displayed to have sizes in conformity with DICOM standard, or the test patterns can be displayed to have sizes so that the visual recognition rate may be improved according to the visual characteristic of the observer.

### Second Embodiment

In a second embodiment, an example of changing a plurality of test patterns so that one test pattern is displayed on one screen, and of varying the display position of the test pattern on each changed screen at every change of the screen is described.

First, the configuration thereof is described.

Because the medical image display apparatus in the second embodiment has the same configuration as that of the medical image display apparatus of the first embodiment, their same units are denoted by the same reference marks to be described.

That is, the medical image display apparatus 10 of the second embodiment comprises, as shown in FIG. 1, the control unit 11, the operation unit 12, the display unit 13, the communication unit 14, the RAM 15, and the storage unit 16.

The control unit 11 unwinds various control programs, which are stored in the storage unit 16, such as a system program, a first pattern display processing program according to the present invention, and a calibration processing program, to the RAM 15, and performs the centralized control of the operation of each unit in conformity with the control program.

In the first pattern display processing, the level range of the drive levels at which the medical image display apparatus 10 can perform display drives is divided into N equal parts, and N test patterns to each of which each drive level, one of the divided N equal parts, is set are outputted to the display unit 13 together with the information of their drive levels, and each test pattern is driven at the set drive level to be displayed. That is, each test pattern is displayed at the luminance level according to each drive level, one of the divided N equal parts. Each test pattern comprises two display regions to which different drive levels are severally set in order that each test pattern is displayed at a luminance level different from each other.

When the N test patterns are displayed on the display unit 13, the control unit 11 generates screen data of locating one test pattern on one screen, and outputs the generated screen data to the display unit 13 together with the information of the drive level set to the test pattern. Thereby, the control unit 11 changes the test patterns one by one to each screen so as to display one test pattern on one screen. Moreover, the control unit 11 generates screen data of moving each test pattern displayed on each changed screen every change of the screens to locate the test patterns at different display positions, and makes the display positions vary over each changed screen. That is, a control section can be realized by the cooperation of the control unit 11 and the first pattern processing program.

In the calibration processing, when an adjustment operation of the luminance level has been performed about the displayed test pattern through the operation unit 12, the control unit 11 alters the drive level of the test pattern according to the operation instruction to make the display unit 13 perform a display drive. When the adjustment operation has ended, the control unit 11 calculates a correction curve for correcting the display gradation characteristic of the medical image display apparatus 10 based on the values of the drive levels of the two display regions constituting each test pattern. Then, the control unit 11 creates a LUT in which output values to input values of the correction curve are prescribed, and stores the LUT in the storage unit 16.

The operation unit 12 includes a keyboard and a mouse. When the keyboard or the mouse is operated, the operation unit 12 generates an operation signal according to the operation, and outputs it to the control unit 11. Incidentally, the operation unit 12 may include a touch panel which is integrally formed with the display unit 13, and the like.

The display unit 13 is a display section having a monitor such as an LCD and a CRT, and drives the monitor in conformity with the control of the control unit 11 to display various kinds of screen data such as an operation screen and the display screens of the test patterns. Incidentally, when the display unit 13 displays each test pattern [n], the display unit 13 performs the display drive of the monitor at the specified drive level in conformity to the drive level information inputted together with the test pattern [n].

The communication unit 14 includes communication interfaces such as a NIC, a modem, and a router, and performs data communication with external apparatuses on a LAN provided in a hospital through the communication interfaces. For example, the communication unit 14 accesses an image server managing medical images of patients made to be a database to obtain the data of a medical image.

The RAM 15 forms a work area which temporarily stores various programs to be executed by the control unit 11, the data processed by these programs, and the like.

The storage unit 16 comprises a magnetic or an optical recording medium, a semiconductor memory, or the like, and stores various control programs such as a system program and the calibration processing program. Moreover, the storage unit 16 stores the information such as the parameters of movement amounts at the time of moving the display positions of the test patterns [n] over each changed screen, the parameters of movable region ranges, and the display screen sizes of the display unit 13 as parameters used at the time of calibration processing. Moreover the storage unit 16 stores, for example, a LUT for correcting the display gradation characteristic of the medical image display apparatus 10 created by the calibration processing as a processing result of a program.

Moreover, the storage unit 16 stores the data of a plurality of test patterns used in the calibration processing.

Incidentally, because the same test patterns as those shown in FIGS. 2-4 in the first embodiment can be applied, the detailed descriptions concerning the test patterns are omitted here.

Next, the first pattern display processing and the calibration processing executed by the medical image display apparatus 10 of the present embodiment are described using the test patterns [n].

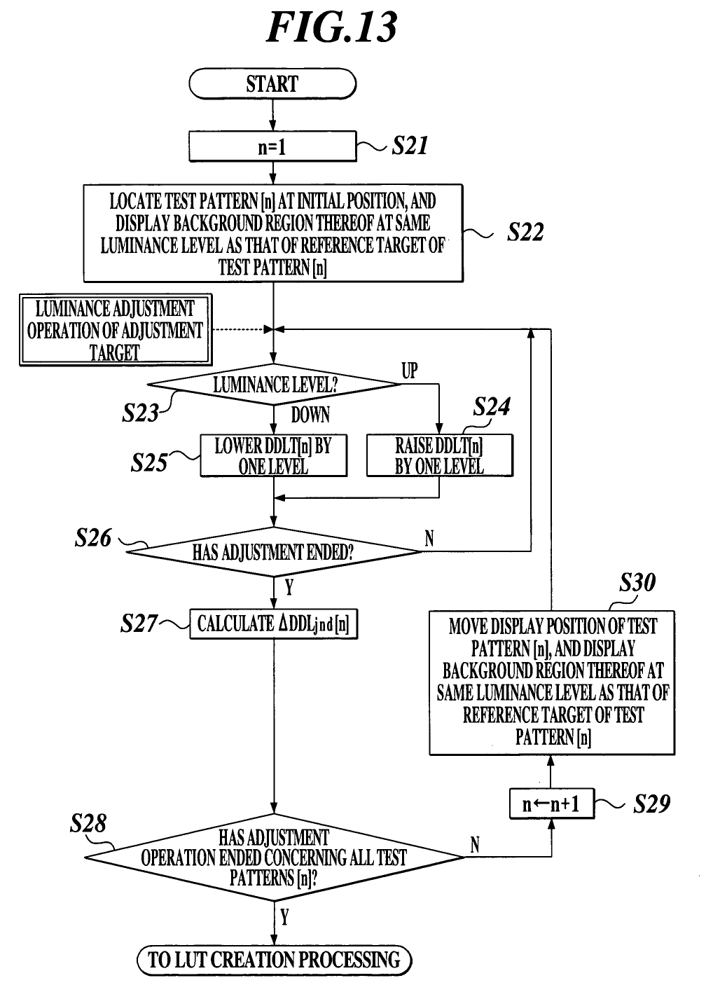

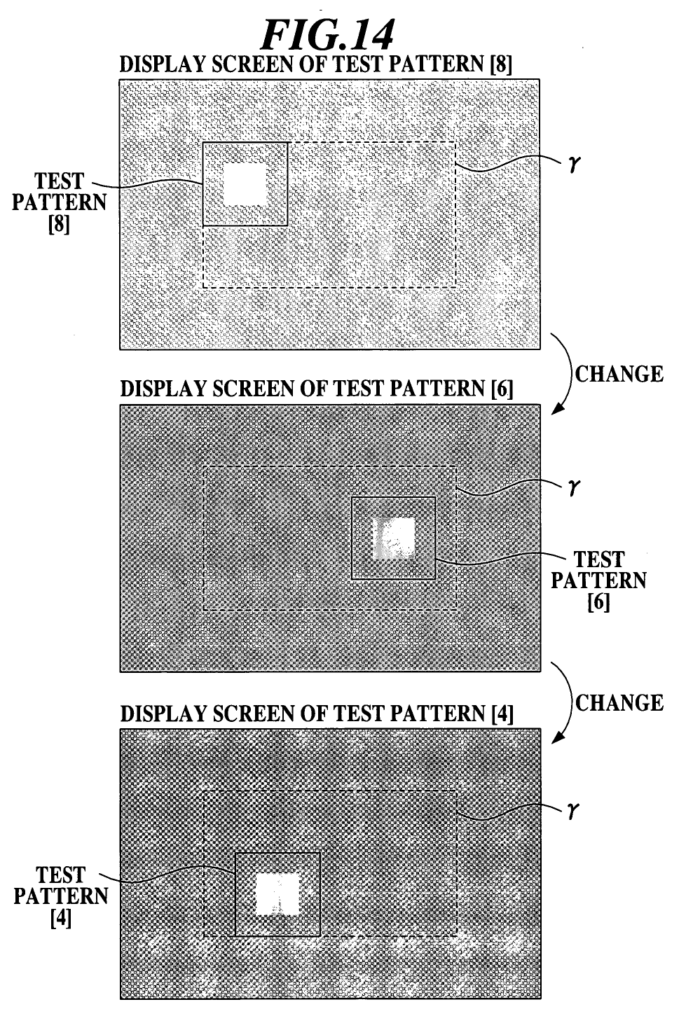

FIG. 13 is a flowchart illustrating the first pattern display processing, and FIG. 14 is a view showing transitions of the display screens displayed at the time of the processing. The processing is started when the calibration is selected in an environment setting menu.

In the first pattern display processing shown in FIG. 13, the pattern number n of a test pattern to be displayed is first set to n=1, which is an initial value (Step S21). When the pattern number n has been set, in the control unit 11, the data of the test pattern [n] having the pattern number n is read from the storage unit 16. Then, display screen data is generated. The display screen data indicates the location of the test pattern [n] at a predetermined initial position on the display screen and the setting of the drive level of the background region of the test pattern [n] to be the same level as the drive level DDLK [n] of the reference target. The generated display screen data is outputted to the display unit 13 together with the drive level information of each of the display regions. In the display unit 13, the display drive of the monitor is performed in conformity with the drive level information, and the display screen including the test pattern [n] is displayed (Step S22).

FIG. 14 shows examples of the display screens of test patterns [8], [6], and [4]. When the test pattern [6] is described as an example, because only one pattern is displayed on one screen and the background region thereof is driven to be displayed by the same drive level as that of the reference target, the background region is displayed at the same luminance level as that of the display region of the reference target. Moreover, the test pattern [6] is located at a position previously set as an initial position, and is displayed there. Incidentally, dotted lines in the figures indicate previously set region ranges r as regions in which the test patterns [n] can be moved, and the dotted lines do not actually displayed on the display screens.

When a test pattern [n] has been displayed in such a way, the operator performs an adjustment operation of raising or lowering the luminance level of an adjustment target until the luminance level difference between the adjustment target and the reference target in the test pattern [n] becomes the minimum luminance level difference which can be visually recognized by visual observation through the operation unit 12. The minimum luminance level difference which can be visually recognized means a luminance level difference at the limit of visually recognizing the luminance level difference as a result of an operator's adjusting the luminance level of the adjustment target in accordance with the luminance level of the reference target. To put it concretely, the luminance level is adjusted until a stage at which the operator would be unable to identify the luminance level difference between the adjustment target and the reference target if the luminance level of the adjustment target were lowered by more one level.

In the medical image display apparatus 10, when an operation of luminance adjustment of an adjustment target is made through the operation unit 12, the signal analysis of the operation signal is performed, and it is distinguished which the operation instruction is directed to raising the luminous level or lowering the luminous level (Step S23). When the operation instruction is directed to raising the luminance level (Step S23; up), the drive level DDLT [n] of the adjustment target is increased by one level, and the adjustment target during being displayed is driven to be displayed at the increased drive level DDLT (Step S24). On the other hand, when the operation instruction is performed in order to lower the luminance (Step S23; down), the drive level DDLT [n] is lowered by one level, and the adjustment target during being displayed is driven to be displayed at the lowered drive level DDLT [n] (Step S25). That is, in response to one operation, a display is performed at a luminance level raised or lowered by one drive level. Incidentally, even when the operation of lowering the luminance level of the adjustment target is repeated, the adjustment operation is made to be restricted lest the luminance level of the adjustment target should be equal to the luminance level of the reference target or less by invalidating the adjustment operation of making the luminance level equal to the drive level of the reference target or less, or by a similar measure.

When the adjustment operation of the drive level has been performed, it is judged whether the adjustment operation has ended or not (Step S26). When the adjustment operation is still being performed through the operation unit 12 (Step S26; N), the processing returns to the process of Step S23, and the luminance level is adjusted in conformity with the inputted operation instruction. On the other hand, when the adjustment operation has ended (Step S26; Y), a drive level DDLT′ [n] of the adjustment target of the test pattern [n] at the time of an adjustment end is judged, and a difference ΔDDL_{jnd} [n] between the drive level DDLT′ [n] and the drive level DDLK [n] of the reference target is calculated (Step S27). The difference ΔDDL_{jnd} [n] corresponds to the minimum luminance level difference which the operator can visually recognize in the test pattern [n].

Subsequently, it is judged whether the adjustment operation mentioned above has ended about all test patterns [n] or not (Step S28). When the adjustment operation has not ended about all of the test patterns [n] (Step S28; N), the pattern number n is incremented by only one (Step S29). Then, the data of the test pattern [n] of the incremented pattern number n is read from the storage unit 16, and display screen data is generated. The display screen data indicates the location of the test pattern [n] to a position moved from the display position of the test pattern [n] which is now being displayed by a predetermined quantity and the setting of the drive level of the background region of the test pattern [n] to the same level as the drive level DDLK [n] of the reference target of the test pattern [n]. Incidentally, the display position of the test pattern [n] is supposed to move in a predetermined region range r.

The generated display screen data is outputted to the display unit 13 together with the drive level information of each of the display regions. In the display unit 13, the display drive of the monitor is performed in conformity with the drive level information, and the display screen data including the test pattern [n] is displayed (Step S30). In such a way, when the test pattern [n] of the next pattern number n is displayed, the processing returns to the process of Step S23, and the adjustment operation of the test pattern [n] is repeated.

That is, only one test pattern [n] is displayed on one screen in the display unit 13, as shown in FIG. 14, and each test pattern [n] is sequentially changed to be displayed in the order of the pattern number. Moreover, the display position of the test pattern [n] is moved by the predetermined amount every change of the screen, and the display positions vary over the respective changed screens.

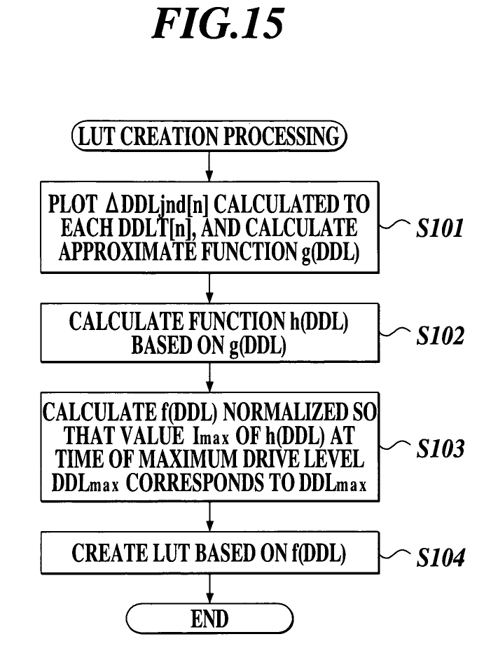

Then, when all the test patterns [n] have been displayed and the adjustment operation has ended Step S28; Y), the processing shifts to the LUT creation processing shown in FIG. 15.

In the LUT creating processing shown in FIG. 15, as shown in FIG. 9, the difference ΔDDL_{jnd} [n] calculated to the drive level DDLK [n] of the reference target of each test pattern [n] is plotted, and the approximate function g(DDL) of each plotted point is calculated (Step S101). That is, each difference ΔDDL_{jnd} [n], which is a discrete value, is interpolated, and a continuous value corresponding to the drive level DDL of all level ranges is obtained. That is, the approximate function g(DDL) is a function showing a correspondence relation between the drive level DDL and the minimum drive level difference ΔDDL_{jnd} [n].

Subsequently, a function h(DDL) of the integral of the calculated function g(DDL) to every drive level DDL is calculated by the formula 3 (Step S102). The above function g(DDL) expresses the inclination of the function h(DDL).

FIG. 10 is a view showing the function h(DDL). The X-axis thereof expresses the drive level DDL, and the Y-axis thereof expresses the function h(DDL) to the drive level DDL. In this case, supposing that a value of the function h(DDL) corresponding to the maximum drive level DDLₘₐₓ which can be driven by the medical image display apparatus 10 is set to Iₘₐₓ, the function h(DDL) (i.e. the values of the Y-axis) is normalized so that the value Iₘₐₓ corresponds to the maximum drive level DDLₘₐₓ, and a function f(DDL) expressing the normalized function h(DDL) is calculated by the formula 4 (Step S103).

Then, a curve shown in FIG. 11 is created. The curve is obtained by substituting the drive levels DDL, which are the units of the X-axis of FIG. 10, with the input levels of image values (Pₘₐₓ in the figure shows the maximum gradation which can be expressed by the medical image display apparatus 10), and by substituting the function h(DDL), which is the unit of the Y-axis, with the calculated function f(DDL). With this curve, a drive level which realizes a corresponding luminance level from the input level of a pixel value can be obtained so that the input level of the pixel value may be finally displayed at a luminance level visually linear to the input level of the pixel value. That is, the curve is a correction curve for correcting the display gradation characteristic according to the display characteristic of the medical image display apparatus 10 (that is, for correcting a relation between the input value of a pixel value and a luminance level at the time of being actually displayed according to the input level). In the control unit 31, the output values to the input values of this correction curve are calculated and are tabled to create a LUT (Step S104). When the created LUT is stored in the storage unit 16 as the LUT for correcting the display gradation characteristic of the medical image display apparatus 10, the present processing ends.

When a medical image is displayed, the created LUT is referred to, and the drive level (output value) corresponding to the input level (input value) of the pixel value of the medical image is obtained to perform a display and a drive at the drive level. Because the drive level obtained from the LUT is a drive level corrected in order that the luminance level at the time of being driven to be displayed may be in a visually linear relation with the input level of the pixel value, the drive and the display mean that the display gradation characteristic of the medical image display apparatus 10 is corrected according to the display characteristic of the medical image display apparatus 10 and the visual characteristic of the operator.

As mentioned above, according to the second embodiment, when the test patterns for calibration are displayed, the test patterns are changed to be displayed so that one pattern is displayed on one screen. Consequently, a fall of the accuracy of the visibility owing to the entering of a test pattern other than the test pattern to be visually recognized into the visual field of an operator can be prevented. Consequently, more accurate calibration can be performed.

Moreover, because the display positions of the test patterns [n] are moved every change of the screens, even when the afterimage of the test pattern [n] displayed on the preceding screen is generated, the test pattern [n] in the next screen is displayed at a position different from the display position of the test pattern [n] on the preceding screen. Consequently, it is possible to prevent the test pattern [n] which is being visually recognized by an operator from being influenced by the afterimage of the test pattern [n] on the preceding screen.

Moreover, in each changed screen, because the background region of the test pattern [n] is driven at the same drive level as the drive level DDLK [n] of the reference target of the test pattern [n], the background region and the reference target are displayed at the same luminance level. Thus, the luminance level of the test pattern [n] and the luminance level of the background region are made to correspond to each other, and consequently the eyes of the operator adapt themselves to the luminance level, and the accuracy of the visibility is improved. Incidentally, although the drive levels of the background regions are made to be the ones same as the drive levels DDLK [n] of the reference targets in the present embodiment, as long as the luminance levels of the background regions vary according to the luminance levels of the test patterns [n], the drive levels of the background regions may be set to be other values such as average values of the drive levels DDLK [n] of the reference targets and the drive levels of the DDLT [n] of the adjustment targets or a set value in the range of from the drive levels DDLK [n] to the drive levels DDLT [n].

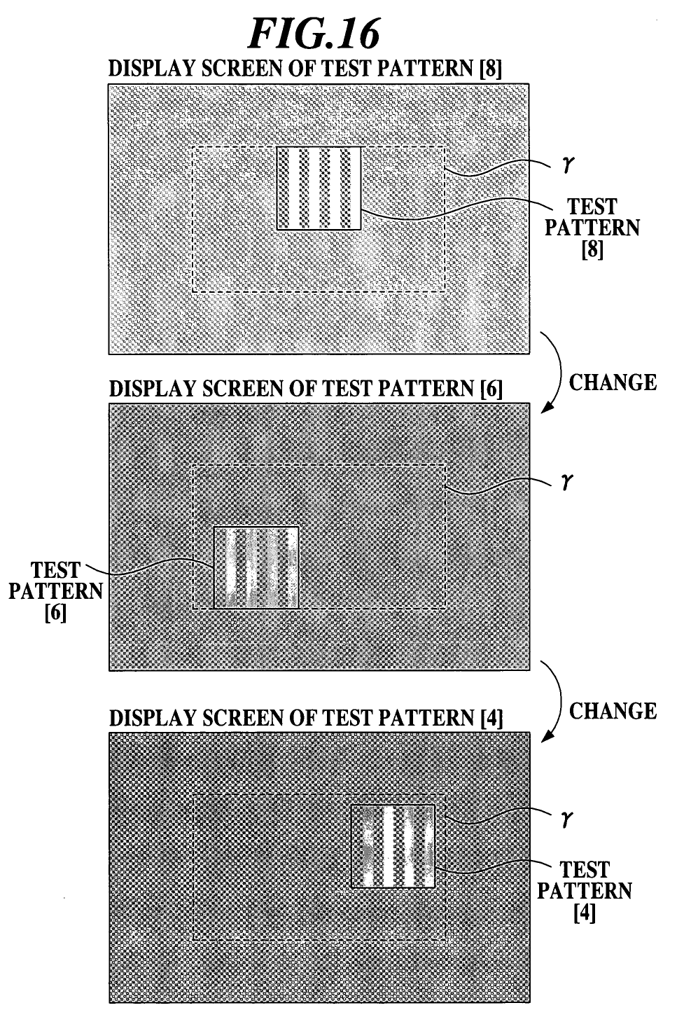

Moreover, although the example using the rectangular patterns is described in the present embodiment, similar processing is performed also when line patterns are used. Screen transition views in the case where the line patterns are used are shown in FIG. 16. Like in the case of the rectangular patterns, the line patterns are changed so that one line pattern may be displayed on one screen, and the display positions of the line patterns are moved every change of the screens.

### MODIFIED EXAMPLE 1 OF SECOND EMBODIMENT

Hereinafter, an example of changing a plurality of test patterns so that one test pattern is displayed on one screen, and of varying the display position of the test pattern in each changed screen.

First, the configuration thereof is described.

Because the medical image display apparatus in modified example 1 has the substantially same configuration as that of the medical image display apparatus 10 of the second embodiment, the illustration of the configuration is omitted. The same components thereof are denoted by the same reference marks, and only different functional portions are described. That is, the medical image display apparatus 10 of the modified example 1 comprises the control unit 11, the operation unit 12, the display unit 13, the communication unit 14, the RAM 15, and the storage unit 16.

The control unit 11 reads a second pattern display processing program according to the present invention from the storage unit 16, and performs the centralized control of the processing operation of each unit in conformity with the program.

In the second pattern display processing, when the N test patterns [n] are displayed on the display unit 13, the control unit 11 outputs screen data indicating the locating of one test pattern [n] to one screen to the display unit 13 together with the information of the drive level set to the test pattern. Thereby, the control unit 11 displays one of the test patterns on one screen in the way of changing the test pattern every change of the screens. Moreover, the control unit 11 moves the display position of the test pattern [n] to be displayed on each changed screen at a predetermined speed, and thereby varies the display position. That is, a control section can be realized by the cooperation of the control unit 11 and the second pattern processing program.

The storage unit 16 stores a second pattern display processing program. Moreover, the storage unit 16 stores the information of a speed (0.5 cm/s in the present embodiment) at the time of moving the display position of the test pattern [n] on each changed screen, the information of movable region ranges, and the like.

Next, the operation of the medical image display apparatus 10 of the modified example 1 is described.

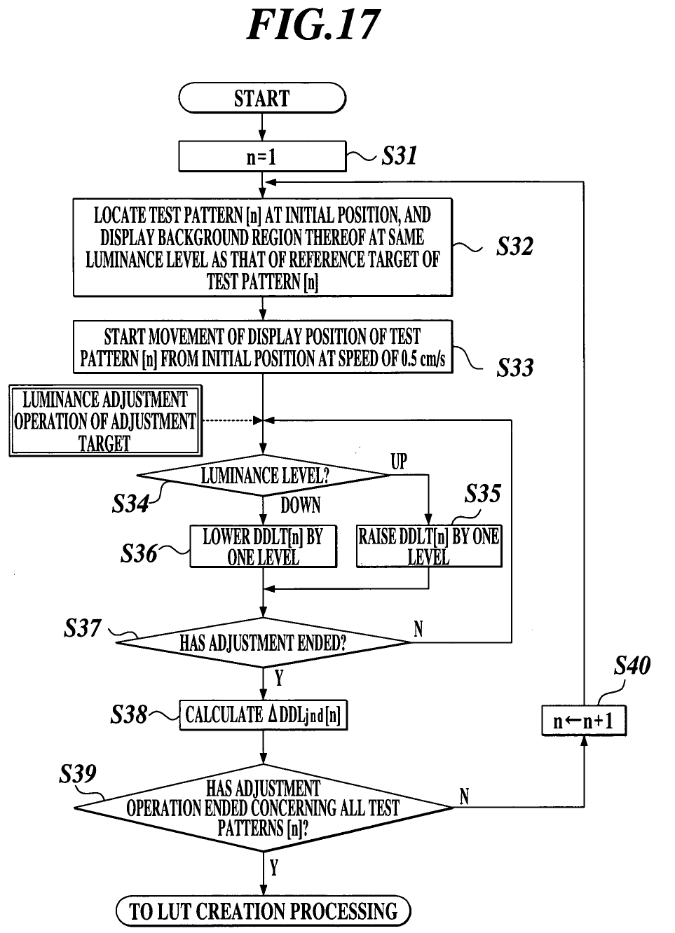

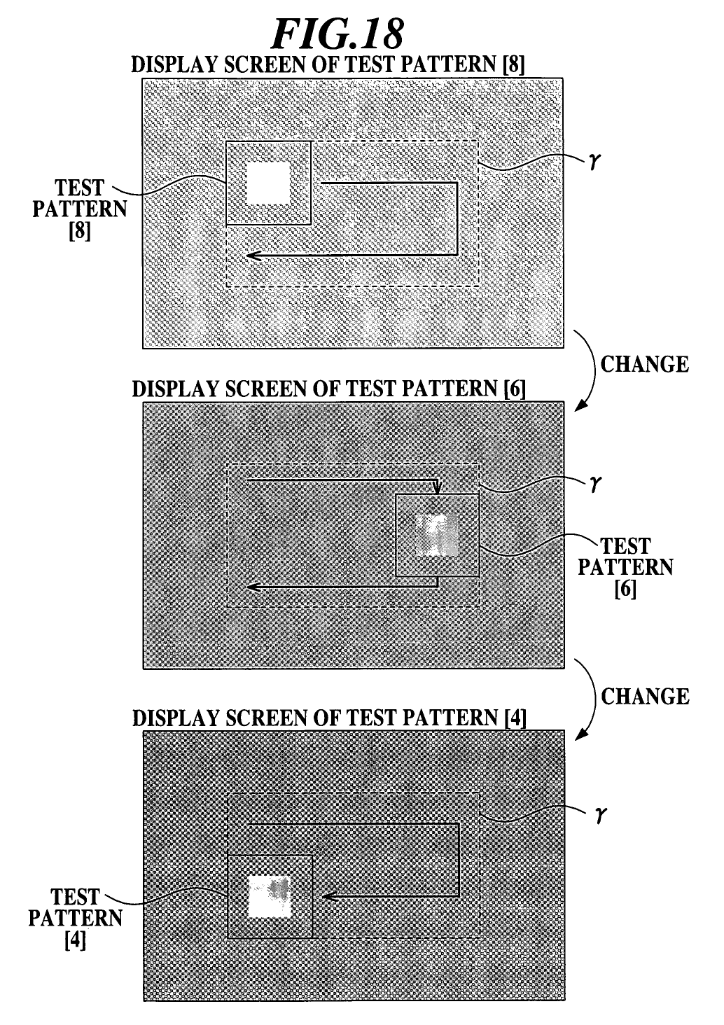

FIG. 17 is a flowchart illustrating the second pattern display processing executed by the medical image display apparatus 10, and FIG. 18 is a view showing screen transitions of the display screens at the time of the processing.

In the second pattern display processing shown in FIG. 17, the pattern number n of a test pattern to be displayed is first set to n=1, which is an initial value (Step S31). When the pattern number n has been set, the data of the test pattern [n] having the pattern number n is read from the storage unit 16. Then, the test pattern [n] is located at a predetermined initial position on the display screen, and display screen data indicating the setting of the drive level of the background region of the test pattern [n] to be the same level as the drive level DDLK [n] of the reference target of the test pattern [n] is generated. The generated display screen data is outputted to the display unit 13 together with the drive level information of each of the display regions. In the display unit 13, the display drive of the monitor is performed in conformity with the drive level information, and the display screen of the test pattern [n] is displayed (Step S32).

FIG. 18 is a view showing examples of the display screens of test patterns [8], [6], and [4]. When the test pattern [6] is described as an example, the test pattern [6] is located at a position previously determined as an initial position on the display screen, and is displayed there. Because the background region of the test pattern [n] is driven to be displayed at the same drive level as the drive level DDLK [n] of the reference target, the background region thereof is displayed at the same luminance level as that of the reference target. Incidentally, dotted lines in the figures indicate previously set region ranges r as regions in which the test patterns [n] can be moved, and the dotted lines do not actually displayed on the display screens.

When the test pattern [n] is displayed, the movement of the display position of the test pattern [n] is started at the speed of 0.5 cm/s from the displayed initial position (Step S33). Incidentally, the test pattern [n] is moved in the region r.

When the test pattern [n] has been displayed in such a way, the operator performs an adjustment operation of raising or lowering the luminance level of an adjustment target until the luminance level difference between the adjustment target and the reference target in the test pattern [n] becomes the minimum luminance level difference which can be visually recognized by visual observation through the operation unit 12. Incidentally, the test pattern [n] on the display screen is moving at a constant speed during the adjustment operation.

In the medical image display apparatus 10, when an operation of luminance adjustment of an adjustment target is made through the operation unit 12, the signal analysis of the operation signal is performed, and it is distinguished which the operation instruction is directed to raising the luminous level or to lowering the luminous level (Step S34). When the operation instruction is directed to raising the luminance level (Step S34; up), the drive level DDLT [n] of the adjustment target is increased by one level, and the adjustment target during being displayed is driven to be displayed at the increased drive level DDLT (Step S35). On the other hand, when the operation instruction is performed in order to lower the luminance (Step S34; down), the drive level DDLT [n] is lowered by one level, and the adjustment target during being displayed is driven to be displayed at the lowered drive level DDLT [n] (Step S36). Because the adjustment operation is the same as that at the time of the first pattern processing described above, the details thereof is omitted here.

When the adjustment operation of the drive level has been performed, it is judged whether the adjustment operation has ended or not (Step S37). When the adjustment operation is still being performed through the operation unit 12 (Step S37; N), the processing is return to the process of Step S34, and the luminance level is adjusted in conformity with the inputted operation instruction. On the other hand, when the adjustment operation has ended (step S37; Y), a drive level DDLT′ [n] of the adjustment target of the test pattern [n] at the time of the adjustment end is judged, and a difference ΔDDL_{jnd} [n] between the drive level DDLT′ [n] and the drive level DDLK [n] of the reference target is calculated (Step S38).

Subsequently, it is judged whether the adjustment operation mentioned above has ended about all test patterns [n] or not (Step S39). When the adjustment operation has not ended about all of the test patterns [n] (step S39; N), the pattern number n is incremented by only one (Step S40). Then, the processing returns to the process of Step 32, and the adjustment operation of the next test pattern [n] is repeated. That is, as shown in FIG. 18, only one test pattern [n] is displayed on one screen, and each test pattern [n] is sequentially changed to be displayed in the order of the pattern number thereof.

Then, when all the test patterns [n] have been displayed and the adjustment operation has ended (step S39; Y), the processing shifts to the LUT creation processing shown in FIG. 15. Because the LUT creation processing is performed in the similar way to that of the second embodiment, the description thereof is omitted.

As mentioned above, according to the modified example 1, when the test patterns for calibration are displayed, the test patterns are changed to be displayed so that one pattern is displayed on one screen. Consequently, a fall of the accuracy of the visibility owing to the entering of a test pattern other than the test pattern to be visually recognized into the visual field of the operator can be prevented. Consequently, more accurate calibration can be performed.

Moreover, because the display position of the test pattern [n] displayed on each changed screen is moved on the screen and the display position thereof always changes, it can be prevented that the test pattern [n] displayed on each changed screen remains on the eyes of an operator as an afterimage. Consequently, the influence given to the test pattern [n] displayed on the succeeding screen by the test pattern [n] of the preceding screen can be decreased. Thereby, a fall of the accuracy of the visual recognition of the operator can be prevented.

Moreover, in each changed screen, because the background region of the test pattern [n], is driven at the same drive level as the drive level DDLK [n] of the reference target of the test pattern [n], the background region and the reference target are displayed at the same luminance level. Thus, the luminance level of the test pattern [n] and the luminance level of the background region are made to correspond to each other, and consequently, the eyes of the operator adapt themselves to the luminance level, and the accuracy of the visibility is improved. Incidentally, although the drive levels of the background regions are made to be the ones same as the drive levels DDLK [n] of the reference targets in the present embodiment, as long as the luminance levels of the background regions vary according to the luminance levels of the test patterns [n], the drive levels of the background regions may be set to be other values such as average values of the drive levels DDLK [n] of the reference targets and the drive levels of the DDLT [n] of the adjustment targets or a set value in the range of from the drive levels DDLK [n] to the drive levels DDLT [n].

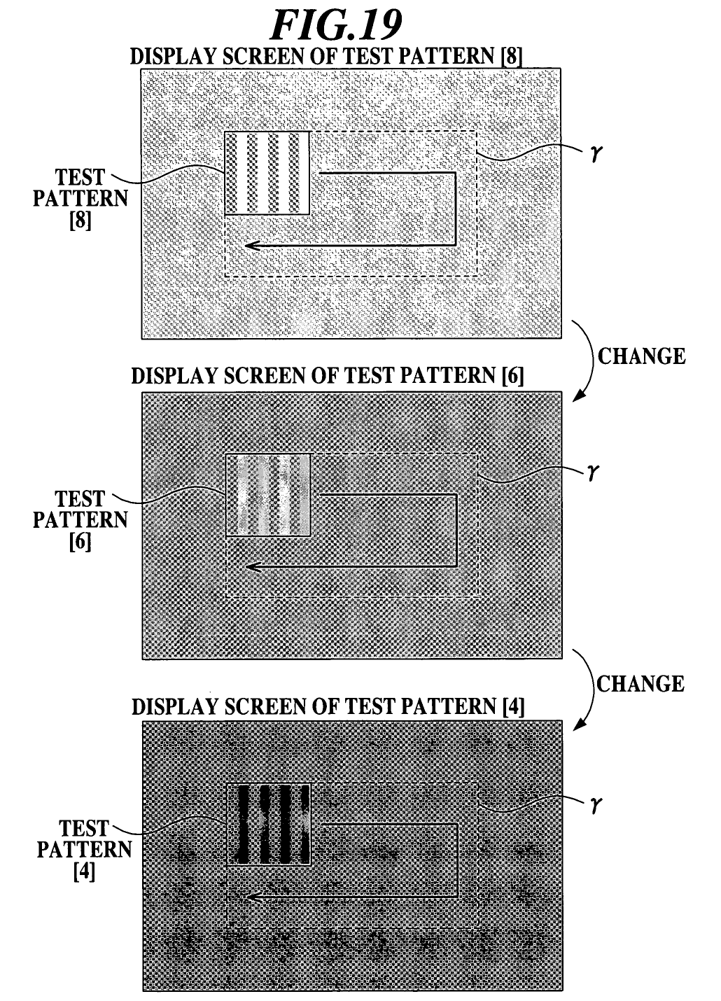

Moreover, although the example using the rectangular patterns is described in the present embodiment, similar processing is performed also when line patterns are used. Screen transition views in the case where the line patterns are used are shown in FIG. 19. Like in the case of the rectangular patterns, the line patterns are changed so that one line pattern may be displayed on each screen, and the display positions of the line patterns are moved every change of the screens.

### MODIFIED EXAMPLE 2 OF SECOND EMBODIMENT

Hereinafter, an example of a display of changing a plurality of test patterns so that one test pattern is displayed on one screen, and of a display of varying the display size of the test pattern in each changed screen.

First, the configuration thereof is described.

Because the medical image display apparatus in modified example 2 has the substantially same configuration as that of the medical image display apparatus 10 of the second embodiment, the illustration of the configuration is omitted. Their same components are denoted by the same reference marks, and only different functional portions are described. That is, the medical image display apparatus 10 of the modified example 2 comprises the control unit 11, the operation unit 12, the display unit 13, the communication unit 14, the RAM 15, and the storage unit 16.

The control unit 11 reads a third pattern display processing program from the storage unit 16, and performs the centralized control of the processing operation of each unit in conformity with the program.

In the third pattern display processing, when the N test patterns [n] are displayed on the display unit 13, the control unit 11 outputs screen data indicating of the locating of one test pattern [n] to one screen to the display unit 13 together with the information of the drive level set to the test pattern. Thereby, the control unit 11 displays one of the test patterns on one screen in the way of changing the test pattern every change of the screens. Moreover, the control unit 11 expands or reduces the display size of the test pattern [n] to be displayed on each changed screen at a predetermined speed, and thereby varies the display size. That is, a control section can be realized by the cooperation of the control unit 11 and the third pattern processing program.

The storage unit 16 stores a third pattern display processing program. Moreover, the storage unit 16 stores the parameter information necessary at the time of the third pattern display processing such as the parameter of a speed (1 cm²/s in the present embodiment) at the time of varying the display size of the test pattern [n] and the parameters of the maximum size to which the display size can be expanded and the minimum size to which the display size can be reduced.

Next, the operation of the medical image display apparatus 10 of the modified example 2 is described.

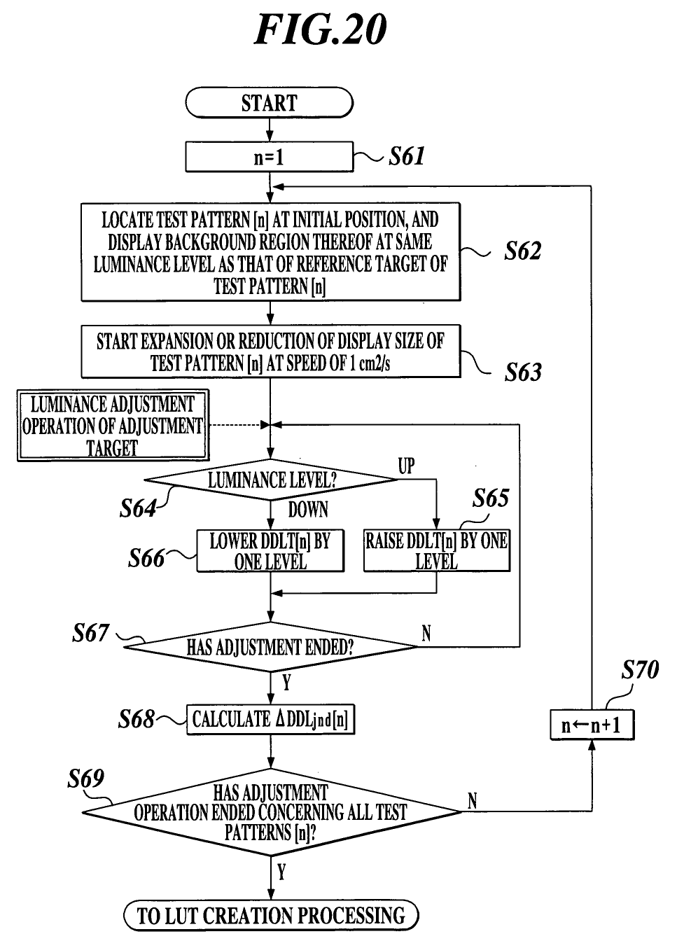

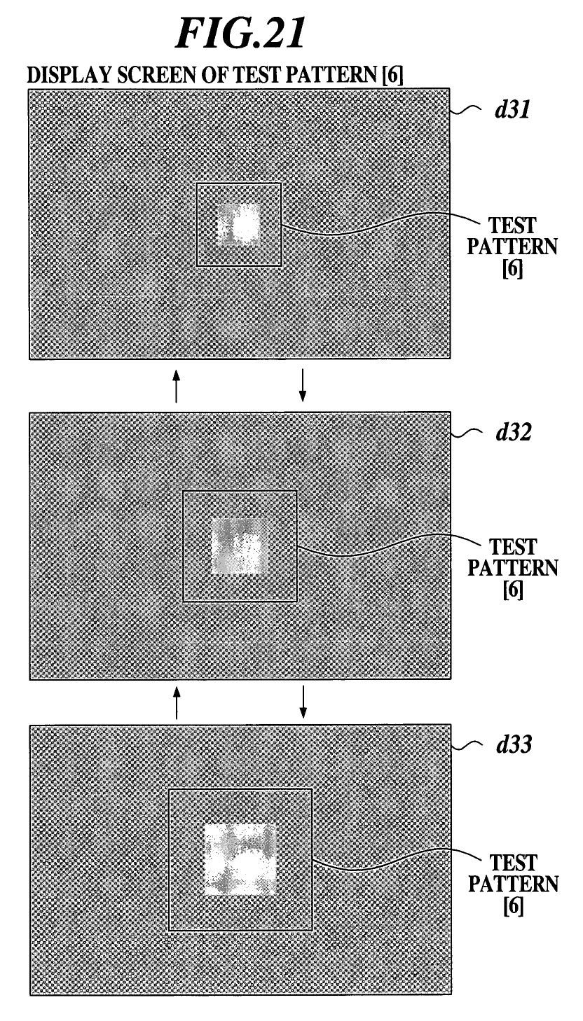

FIG. 20 is a flowchart illustrating the third pattern display processing executed by the medical image display apparatus 10, and FIG. 21 is a view showing examples of display screens displayed at the time of the processing.

In the third pattern display processing shown in FIG. 20, the pattern number n of a test pattern to be displayed is first set to n=1, which is an initial value (Step S61). When the pattern number n has been set, the data of the test pattern [n] having the pattern number n is read from the storage unit 16. Then, the test pattern [n] is located at a predetermined initial position on the display screen, and display screen data indicating the setting of the drive level of the background region of the test pattern [n] to be the same level as the drive level DDLK [n] of the reference target of the test pattern [n] is generated. The generated display screen data is outputted to the display unit 13 together with the drive level information of each of the display regions. In the display unit 13, the display drive of the monitor is performed in conformity with the drive level information, and the display screen of the test pattern [n] is displayed (Step S62).

When the test pattern [n] is displayed, the variations of the display size of the test pattern [n] are started at the speed of 1 cm²/s from the displayed initial position, and expansion or reduction is repeated (Step S63).

FIG. 21 is a view showing the variation of the display screen of the test pattern [6].

As shown in FIG. 21, the test pattern [6] is displayed at a position (the central part of the screen) previously determined as the initial position in the whole display screen. Because the background region of the test pattern [6] is displayed at a drive level same as the drive level DDLK [6] of the reference target, the background region is displayed at the same luminance level as that of the reference target.

The test pattern [6] is gradually expanded as time elapses. When the size of the test pattern [6] reaches the size previously set as the maximum size, the size of the test pattern [6] is gradually reduced from the maximum size. Then, when the size of the test pattern [6] reaches the size previously set as the minimum size, the size of the test pattern [6] is gradually expanded again. That is, when the display screens d31, d32, and d33 are displayed in the order of d31→d32→→d33, the display screens d31, d32, and d33 are reversely displayed in the order of d33→d32→d31 at this time. Then, the expansion or the reduction of the display size is repeated until the luminance adjustment of the test pattern [n] ends.

In such a way, when the test pattern [n] has been displayed, the operator performs an adjustment operation of raising or lowering the luminance level of an adjustment target until the luminance level difference between the adjustment target and the reference target in the test pattern [n] becomes the minimum luminance level difference which can be visually recognized by visual observation through the operation unit 12. Incidentally, the test pattern [n] on the display screen is changing its size at a constant speed during the adjustment operation.

In the medical image display apparatus 10, when an operation of luminance adjustment of an adjustment target is made through the operation unit 12, the signal analysis of the operation signal is performed, and it is distinguished which the operation instruction is directed to raising the luminous level or to lowering the luminous level (Step S64). When the operation instruction is directed to raising the luminance level (Step S64; up), the drive level DDLT [n] of the adjustment target is increased by one level, and the adjustment target during being displayed is driven to be displayed at the increased drive level DDLT (Step S65). On the other hand, when the operation instruction is performed in order to lower the luminance (Step S64; down), the drive level DDLT [n] is lowered by one level, and the adjustment target during being displayed is driven to be displayed at the lowered drive level DDLT [n] (Step S66). Because the adjustment operation is the same as that at the time of the first pattern processing described above, the details thereof is omitted here.

When the adjustment operation of the drive level has been performed, it is judged whether the adjustment operation has ended or not (Step S67). When the adjustment operation is still being performed through the operation unit 12 (Step S67; N), the processing is return to the process of Step S64, and the luminance level is adjusted in conformity with the inputted operation instruction. On the other hand, when the adjustment operation has ended (step S67; Y), a drive level DDLT′ [n] of the adjustment target of the test pattern [n] at the time of the adjustment end is judged, and a difference ΔDDL_{jnd} [n] between the drive level DDLT′ [n] and the drive level DDLK [n] of the reference target is calculated (Step S68).

Subsequently, it is judged whether the adjustment operation mentioned above has ended about all test patterns [n] or not (Step S69). When the adjustment operation has not ended about all of the test patterns [n] (step S69; N), the pattern number n is incremented by only one (Step S70). Then, the processing returns to the process of Step 62, and the adjustment operation of the next test pattern [n] is repeated. That is, only one test pattern [n] is displayed on one screen, and each test pattern [n] is sequentially changed to be displayed in the order of the pattern number thereof.

Then, when all the test patterns [n] have been displayed and the adjustment operation has ended (step S69; Y), the processing shifts to the LUT creation processing shown in FIG. 15. Because the LUT creation processing is performed in the similar way to that of the second embodiment, the description thereof is omitted.

As mentioned above, according to the modified example 2, when the test patterns for calibration are displayed, the test patterns are changed to be displayed so that one pattern is displayed on one screen. Consequently, a fall of the accuracy of the visibility owing to the entering of a test pattern other than the test pattern to be visually recognized into the visual field of the operator can be prevented. Consequently, more accurate calibration can be performed.

Moreover, because the display size of the test pattern [n] displayed on each changed screen is expanded or reduced to be always varied on the screen, it can be prevented that the test pattern [n] displayed on each changed screen remains on the eyes of an operator as an afterimage. Consequently, the influence given to the test pattern [n] displayed on the succeeding screen by the test pattern [n] of the preceding screen can be decreased. Thereby, a fall of the accuracy of the visual recognition of the operator can be prevented.

Incidentally, although the display size of the test pattern [n] is varied in each changed screen in the present embodiment, the test pattern [n] may be displayed in different display size every change of the screen in spite of being a fixed size in each screen.

Moreover, in each changed screen, because the background region of the test pattern [n] is driven at the same drive level as the drive level DDLK [n] of the reference target of the test pattern [n], the background region and the reference target are displayed at the same luminance level. Thus, the luminance level of the test pattern [n] and the luminance level of the background region are made to correspond to each other, and consequently, the eyes of the operator adapt themselves to the luminance level, and the accuracy of the visibility improves. Incidentally, although the drive levels of the background regions are made to be the ones same as the drive levels DDLK [n] of the reference targets in the present embodiment, as long as the luminance levels of the background regions vary according to the luminance levels of the test patterns [n], the drive levels of the background regions may be set to be other values such as average values of the drive levels DDLK [n] of the reference targets and the drive levels of the DDLT [n] of the adjustment targets or a set value in the range of from the drive levels DDLK [n] to the drive levels DDLT [n].

Incidentally, although the example using the rectangular patterns is described in the present embodiment, similar processing is performed also when line patterns are used. That is, like in the case of the rectangular patterns, the line patterns are changed so that one line pattern may be displayed on each screen, and the expansion or the reduction of the line pattern is repeated to vary the size on each changed screen.

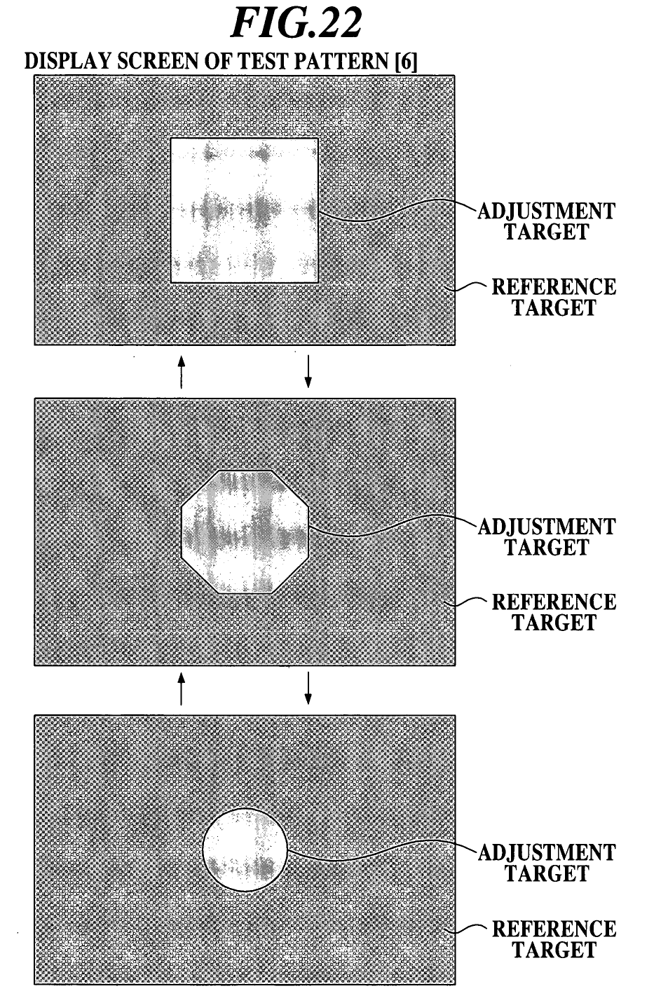

Moreover, although the test pattern [n] is displayed on the display screen at a predetermined ratio and the size thereof is varied in the above description, as shown in FIG. 22, it may be adoptable that the rectangular pattern is displayed on the whole screen and the size and the shape of the adjustment target thereof are varied. In the example shown in FIG. 22, the shape of the display region of the adjustment target is varied from a quadrilateral to a polygon, and then the shape is varied from the polygon to a circular form. Moreover, with the variation of the shape, the size of the adjustment target is also displayed while being reduced. Thereby, the generation of afterimages can be prevented by varying the shape in such a way.

Moreover, the test pattern [n] may be displayed in combination with the second embodiment, and the embodiments of the modified examples 1 and 2 of the second embodiment. For example, in combination with the second embodiment and the embodiment of the modified example 2 of the second embodiment, the display position of the test pattern [n] may be moved every change of the screens, and simultaneously the display size of the test pattern [n] may be always varied in each changed screen.

### Third Embodiment

First, the configuration thereof is described.

Because the medical image display apparatus in a third embodiment has the same configuration as that of the medical image display apparatus 10 of the first embodiment, their same units are denoted by the same reference marks to be described.

That is, the medical image display apparatus 10 of the third embodiment comprises, as shown in FIG. 1, the control unit 11, the operation unit 12, the display unit 13, the communication unit 14, the RAM 15, and the storage unit 16.

The control unit 11 unwinds various control programs such as a system program, which is stored in the storage unit 16, and a calibration processing program according to the present invention, to the RAM 15, and performs the centralized control of the operation of each unit in conformity with the control program.

In the calibration processing, the level range of the drive levels at which the medical image display apparatus 10 can perform display drives is divided into N equal parts, and the data of each of N test patterns to which each drive level, one of the divided N equal parts, is set is outputted to the display unit 13 together with the information of the set drive level to be displayed. That is, each test pattern is displayed at the luminance level according to each drive level, one of the divided N equal parts. Each test pattern comprises two display regions. In one of the display regions, each of the drive levels produced by the division into N equal parts is set, and the other display region is set to have a higher level than each of the drive levels produced by the division into N equal parts by a predetermined level.

When the N test patterns are displayed on the display unit 13, the control unit 11 displays one of the test patterns on one screen in the way of changing the test pattern every screen. Moreover, the control unit 11 alters the size of each display region in which a test pattern is formed according to the size of a display screen in order that a ratio between the area of each display region in which each of the drive levels produced by the division into N equal parts is set and the area of the other display region may be 7:3, namely the area of the display region displayed at a lower luminance level may be larger than the area of the other display region. Then, the control unit 11 makes the display unit 13 display the test pattern the size of which has been altered on the whole display screen of the display unit 13. Incidentally, the ratio of the display areas is not restricted to 7:3, but the ratio is made to be able to be suitably set.

Then, when an adjustment operation of the luminance level has been performed about the displayed test pattern through the operation unit 12, the control unit 11 alters the drive level of the test pattern according to the operation instruction to make the display unit 13 perform a display drive. When the adjustment operation has ended, the control unit 11 obtains a correction curve for correcting the display gradation characteristic of the medical image display apparatus 10 based on the value of the drive level of each line constituting each test pattern. Then, the control unit 11 creates a LUT in which output values to input values of the correction curve are prescribed, and stores the LUT in the storage unit 16.

The operation unit 12 includes a keyboard and a mouse. When the keyboard or the mouse is operated, the operation unit 12 generates an operation signal according to the operation, and outputs it to the control unit 11. Incidentally, the operation unit 12 may include a touch panel which is integrally formed with the display unit 13, and the like.

The display unit 13 includes a monitor such as an LCD and a CRT, and drives the monitor in conformity with a display control signal inputted from the control unit 11. The display unit 13 displays one of the test patterns [n] inputted from the control unit 11 on one screen in the way of changing the test pattern [n] every screen at the time of calibration processing. Incidentally, when the display unit 13 displays each test pattern [n], the display unit 13 performs the display drive of the monitor at the specified drive level in conformity to the drive level information inputted together with the test pattern [n].

The communication unit 14 includes communication interfaces such as a NIC, a modem, and a router, and performs data communication with external apparatuses on a LAN provided in a hospital through the communication interfaces. For example, the communication unit 14 accesses an image server managing medical images of patients made to be a database to obtain the data of a medical image.

The RAM 15 forms a work area which temporarily stores various programs to be executed by the control unit 11, the data processed by these programs, and the like.

The storage unit 16 comprises a magnetic or an optical recording medium, a semiconductor memory, or the like, and stores various control programs such as a system program and a calibration processing program. Moreover, the storage unit 16 stores the information of a ratio (7:3 in the present embodiment) between the display area of the reference target and the display area of the adjustment target, and the information of the size of the display screen of the display unit 13 and the like as the parameters used at the time of the calibration processing. Moreover, the storage unit 16 stores, for example, a LUT for correcting the display gradation characteristic of the medical image display apparatus 10 created by calibration processing as a processing result of a program.

Moreover, the storage unit 16 stores the data of a plurality of test patterns used in the calibration processing.

Incidentally, because the same test patterns as those shown in FIGS. 2 and 3 in the first embodiment can be applied, the detailed descriptions concerning the test patterns are omitted here.

Hereinafter, the calibration processing executed by the medical image display apparatus 10 of the present embodiment using the test patterns [n] is described.

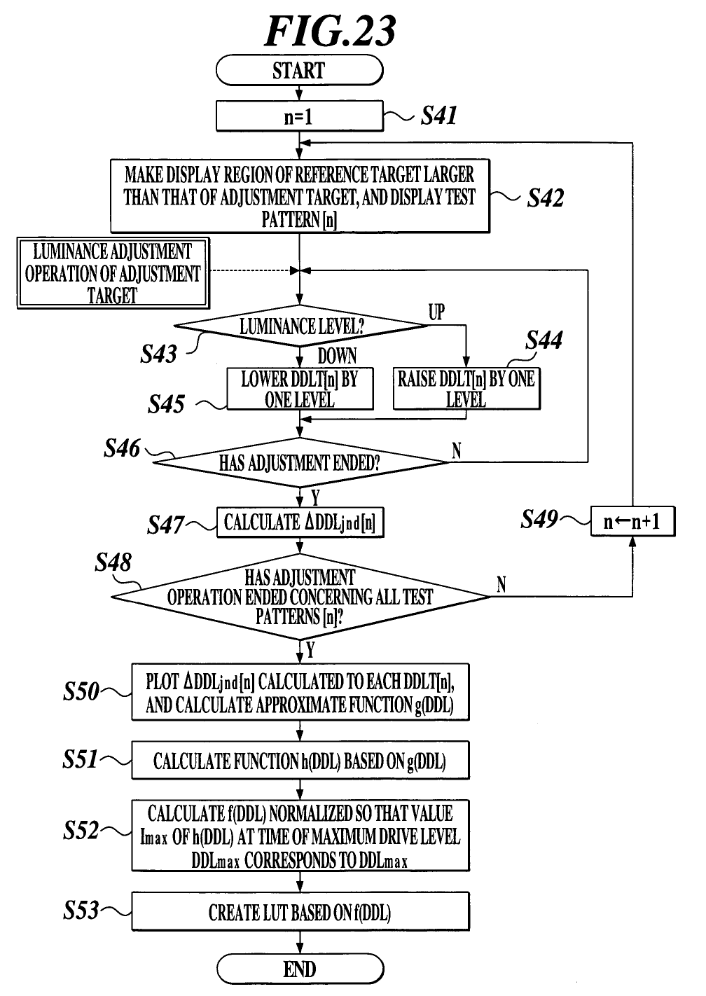

FIG. 23 is a flowchart illustrating the calibration processing. The processing is started when the calibration is selected in an environment setting menu.

In the calibration processing shown in FIG. 23, the pattern number n of a test pattern to be displayed is first set to n=1, which is an initial value (Step S41). Successively, the data of the test pattern [n] is read from the storage unit 16. Then, the size of each display region in the test pattern [n] is adjusted according to the size of the display screen so that the ratio of the area of the display region of the reference target to the area of the display region of the adjustment target is 7:3, and the test pattern [n] the size of which has been adjusted is displayed on the whole display screen of the display unit 13 (Step S42). Incidentally, in the case where the display area of the reference target becomes larger than that of the adjustment target, the display region of the reference target may be located at the inside of the screen and the background thereof may be treated as the display region of the adjustment target. Conversely, the display region of the adjustment target may be located at the inside of the screen, and the display region of the reference target may be located on the background side thereof.

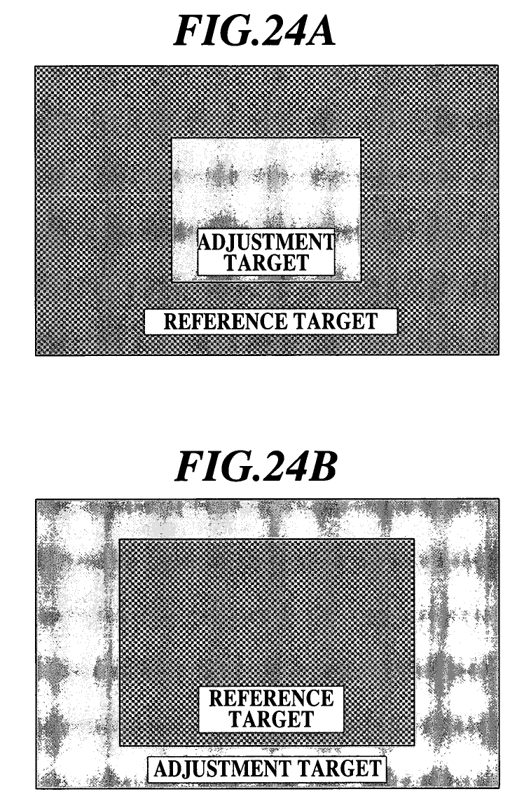

FIGS. 24A and 24B show display examples of the test patterns [n] the sizes of which have been adjusted. FIG. 24A is a view showing a display example of the display region of the reference target located on the background side, and FIG. 24B is a view showing a display example of the display region of the reference target is located in the inside of the screen. In both cases, only one pattern is displayed on one screen.

In such a way, when a test pattern [n] has been displayed, the operator performs an adjustment operation of raising or lowering the luminance level of an adjustment target until the luminance level difference between the adjustment target and the reference target in the test pattern [n] becomes the minimum luminance level difference which can be visually recognized by visual observation through the operation unit 12. The minimum luminance level difference which can be visually recognized means a luminance level difference at the limit of visually recognizing the luminance level difference as a result of an operator's adjusting the luminance level of the adjustment target in accordance with the luminance level of the reference target. To put it concretely, the luminance level is adjusted until a stage at which the operator would be unable to identify the luminance level difference between the adjustment target and the reference target if the luminance level of the adjustment target were lowered by more one level. Incidentally, the operator performing the calibration may be an observer observing the medical image, or may be a maintenance person performing calibration only.

In the medical image display apparatus 10, when an operation of luminance adjustment of an adjustment target is made through the operation unit 12, the signal analysis of the operation signal is performed, and it is distinguished which the operation instruction is directed to raising the luminous level or to lowering the luminous level (Step S43). When the operation instruction is directed to raising the luminance level (Step S43; up), the drive level DDLT [n] of the adjustment target is increased by one level, and the adjustment target during being displayed is driven to be displayed at the increased drive level DDLT (Step S44). On the other hand, when the operation instruction is performed in order to lower the luminance (Step S43; down), the drive level DDLT [n] is lowered by one level, and the adjustment target during being displayed is driven to be displayed at the lowered drive level DDLT [n] (Step S45). That is, in response to one operation, a display is performed at a luminance level raised or lowered by one drive level. Incidentally, even when the operation of lowering the luminance level of the adjustment target is repeated, the adjustment operation is made to be restricted lest the luminance level of the adjustment target should be equal to the luminance level of the reference target or less by invalidating the adjustment operation of making the luminance level equal to the drive level of the reference target or less, or by a similar measure.

When the adjustment operation of the drive level has been performed, it is judged whether the adjustment operation has ended or not (Step S46). When the adjustment operation is still being performed through the operation unit 12 (Step S46; N), the processing returns to the process of Step S44, and the luminance level is adjusted in conformity with the inputted operation instruction. On the other hand, when the adjustment operation has ended (Step S46; Y), a drive level DDLT′ [n] of the adjustment target of the test pattern [n] at the time of an adjustment end is judged, and a difference ΔDDL_{jnd} [n] between the drive level DDLT′ [n] and the drive level DDLK [n] of the reference target is calculated (Step S47). The difference ΔDDL_{jnd} [n] corresponds to the minimum luminance level difference which the operator can visually recognize in the test pattern [n].

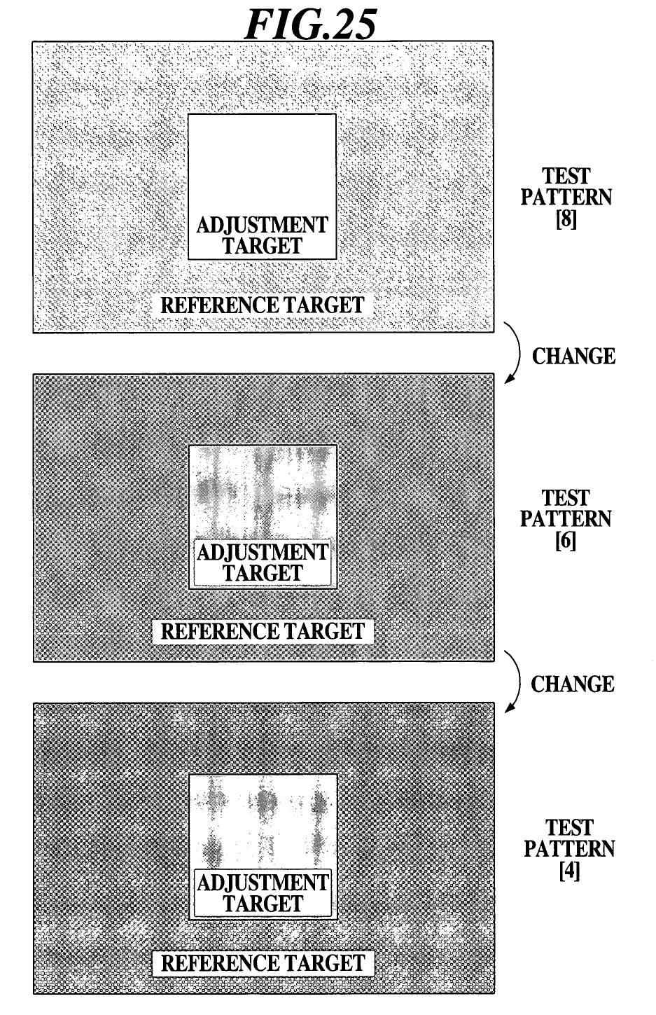

Subsequently, it is judged whether the adjustment operation mentioned above has ended about all test patterns [n] or not (Step S48). When the adjustment operation has not ended about all of the test patterns (Step S48; N), the pattern number n is incremented by only one (Step S49). Then, the processing returns to the process of Step S42, and the adjustment operation of the next test pattern [n] is repeated. That is, only one test pattern [n] is displayed on one screen, and each test pattern [n] is sequentially changed to be displayed in the order of the pattern number, as shown in FIG. 25.

Then, when all the test patterns [n] have been displayed and the adjustment operation has ended (Step S48; Y), as shown in FIG. 9, the difference ΔDDL_{jnd} [n] calculated to the drive level DDLK [n] of the reference target of each test pattern [n] is plotted, and the approximate function g(DDL) of each plotted point is calculated (Step S50). That is, each difference ΔDDL_{jnd} [n], which is a discrete value, is interpolated, and a continuous value corresponding to the drive level DDL of all level ranges is obtained. That is, the approximate function g(DDL) is a function showing a correspondence relation between the drive level DDL and the minimum drive level difference ΔDDL_{jnd} [n].

Subsequently, a function h(DDL) of the integral of the calculated function g(DDL) to every drive level DDL is calculated by the formula 3 (Step S51). The above function g(DDL) expresses the inclination of the function h(DDL).

FIG. 10 is a view showing the function h(DDL). The X-axis thereof expresses the drive level DDL, and the Y-axis thereof expresses the function h(DDL) to the drive level DDL. In this case, supposing that a value of the function h(DDL) corresponding to the maximum drive level DDLₘₐₓ which can be driven by the medical image display apparatus 10 is set to Iₘₐₓ, the function h(DDL) (i.e. the values of the Y-axis) is normalized so that the value Iₘₐₓ corresponds to the maximum drive level DDLₘₐₓ, and a function f(DDL) expressing the normalized function h(DDL) is calculated by the formula 4 (Step S52).

Then, a curve shown in FIG. 11 is created. The curve is obtained by substituting the drive levels DDL, which are the units of the X-axis of FIG. 10, with the input levels of image values, and by substituting the function h(DDL), which is the unit of the Y-axis, with the calculated function f(DDL). With this curve, a drive level which realizes a corresponding luminance level from the input level of a pixel value can be obtained so that the input level of the pixel value may be finally displayed at a luminance level visually linear to the input level of the pixel value. That is, the curve is a correction curve for correcting the display gradation characteristic according to the display characteristic of the medical image display apparatus 10 (that is, for correcting a relation between the input value of a pixel value and a luminance level at the time of being actually displayed according to the input level). In the control unit 31, the output values to the input values of this correction curve are calculated and are tabled to create a LUT (Step S53). When the created LUT is stored in the storage unit 16 as the LUT for correcting the display gradation characteristic of the medical image display apparatus 10, the present processing ends.

When a medical image is displayed, the created LUT is referred to, and the drive level (output value) corresponding to the input level (input value) of the pixel value of the medical image is obtained to perform a display and a drive at the drive level. Because the drive level obtained from the LUT is a drive level corrected in order that the luminance level at the time of being driven to be displayed may be in a visually linear relation with the input level of the pixel value, the drive and the display mean that the display gradation characteristic of the medical image display apparatus 10 is corrected according to the display characteristic of the medical image display apparatus 10, and the visual characteristic of the operator.

As mentioned above, according to the third embodiment, when the test patterns for calibration are displayed, the test patterns are changed to be displayed so that one pattern is displayed on one screen. Consequently, a fall of the accuracy of the visibility owing to the entering of a test pattern other than the test pattern to be visually recognized into the visual field of an operator can be prevented. Consequently, more accurate calibration can be performed.

Moreover, because the reference target, which is a display region having a lower luminance level, is made to have a display area larger than that of the display region of the adjustment target in the test pattern [n], the ratio of the area occupied by the display region of a low luminance level can be enlarged on the display screen. Thereby, the pupils of the operator become easy to open and sensitive to light. Consequently, visual recognition power to a luminance level is improved. Thus, it becomes possible to perform a more accurate calibration.

### Fourth Embodiment

First, the configuration thereof is described.

Because the medical image display apparatus in a fourth embodiment has the same configuration as that of the medical image display apparatus 10 of the first embodiment, their same units are denoted by the same reference marks to be described.

That is, the medical image display apparatus 10 of the fourth embodiment comprises, as shown in FIG. 1, the control unit 11, the operation unit 12, the display unit 13, the communication unit 14, the RAM 15, and the storage unit 16.

The control unit 11 unwinds various control programs such as a system program, which is stored in the storage unit 16, and a calibration processing program according to the present invention, to the RAM 15, and performs the centralized control of the operation of each unit in conformity with the control program.

In the calibration processing, the level range of the drive levels at which the medical image display apparatus 10 can perform display drives is divided into N equal parts, and each of N test patterns to which each drive level, one of the divided N equal parts, is set is outputted to the display unit 13 together with the information of the set drive level to be displayed. Thereby, each test pattern is driven to be displayed at the set drive level. That is, each test pattern is displayed at the luminance level according to each drive level, one of the divided N equal parts. Each test pattern comprises a plurality of pairs of line-like regions (which are referred to line pairs) to each of which a different drive level is set in order that the line pair may be displayed at different luminance levels.

When the N test patterns are displayed on the display unit 13, the control unit 11 outputs screen data indicating the locating of one test pattern [n] to one screen to the display unit 13 together with the information of the drive level set to the test pattern. Thereby, the control unit 11 displays one of the test patterns on one screen in the way of changing the test pattern every change of the screens. Moreover, the control unit 11 reads the information of the visual characteristic and the observation condition of an observer, which have been previously set and stored in the storage unit 16, and the control unit 11 determines the line pair density in the test pattern based on the read information (the method of determining the line pair density will be described later). Then, the control unit 11 outputs the information of the line pair density to the display unit 13 as the information of a display condition of the test pattern. Furthermore, the control unit 11 makes the display unit 13 display each test pattern at the line pair density determined on each changed screen. Moreover, the control unit 11 makes the display unit 13 drive the background region of each test pattern to be displayed at the drive level of 20% of the maximum drive level on each changed screen. The display condition of the background region is in conformity with the digital imaging and communication in medicine (DICOM) standard. That is, a control section can be realized by the cooperation of the control unit 11 and the calibration processing program.

Then, when an adjustment operation of the luminance level the displayed test pattern has been performed through the operation unit 12, the control unit 11 alters the drive level of the test pattern according to the operation instruction to make the display unit 13 perform a display drive. When the adjustment operation has ended, the control unit 11 obtains a correction curve for correcting the display gradation characteristic of the medical image display apparatus 10 based on the value of the drive level of each line constituting each line pair of each test pattern. Then, the control unit 11 creates a LUT in which output values to input values of the correction curve are prescribed, and stores the LUT in the storage unit 16.

The operation unit 12 has a keyboard and a mouse. When the keyboard or the mouse is operated, the operation unit 12 generates an operation signal according to the operation, and outputs it to the control unit 11. Incidentally, the operation unit 12 may include a touch panel which is integrally formed with the display unit 13, and the like.

The display unit 13 is a display section including a monitor such as an LCD and a CRT, and drives the monitor in conformity with a display control signal inputted from the control unit 11. Incidentally, when the display unit 13 displays each test pattern [n], the display unit 13 performs the display drive of the monitor at the specified drive level in conformity to the drive level information inputted together with the test pattern [n].

The communication unit 14 includes communication interfaces such as a NIC, a modem, and a router, and performs data communication with external apparatuses on a LAN provided in a hospital through the communication interfaces. For example, the communication unit 14 accesses an image server managing medical images of patients made to be a database to obtain the data of a medical image.

The RAM 15 forms a work area which temporarily stores various programs to be executed by the control unit 11, the data processed by these programs, and the like.

The storage unit 16 comprises a magnetic or an optical recording medium, a semiconductor memory, or the like, and stores various control programs such as a system program and a calibration processing program. Moreover, the storage unit 16 stores the information such as the observation condition of the observer set through the operation unit 12 in advance (such as an observation distance from the observer to the display screen and the pattern size of the test pattern to be observed. It is supposed here that the observation distance is set to be 500 mm, and the pattern size is set to be 17.5 mm×17.5 mm), the information of visual characteristic of the observer (such as a line pair number per view angle when the visual recognition rate is highest. It is supposed that the line pair number is set to be four line pairs per view angle here.), and the display screen size of the display unit 13, as the parameters to be used at the calibration processing. Moreover, the storage unit 16 stores, for example, a LUT for correcting the display gradation characteristic of the medical image display apparatus 10 created by calibration processing as a processing result of a program.

Incidentally, the observer is a person who observes a medical image when the medical image is displayed, and the observer may be the same person or other person as the operator who performs calibration. Moreover, in the present embodiment, the set visual characteristic of an observer is a generic characteristic of a person who performs such an observation. Then, it is supposed that the display condition of a test pattern [n] is determined based on the generic visual characteristic, but the display condition of the test pattern [n] may be determined according to a visual characteristic of an individual observer after obtaining the visual characteristic of the individual observer by measurements.

Moreover, the storage unit 16 stores the data of a plurality of test patterns used in the calibration processing.

Incidentally, because the same test patterns as those shown in FIGS. 4 and 5 in the first embodiment can be applied, the detailed descriptions concerning the test patterns are omitted here.

Hereinafter, the calibration processing executed by the medical image display apparatus 10 of the present embodiment is described using the test patterns [n].

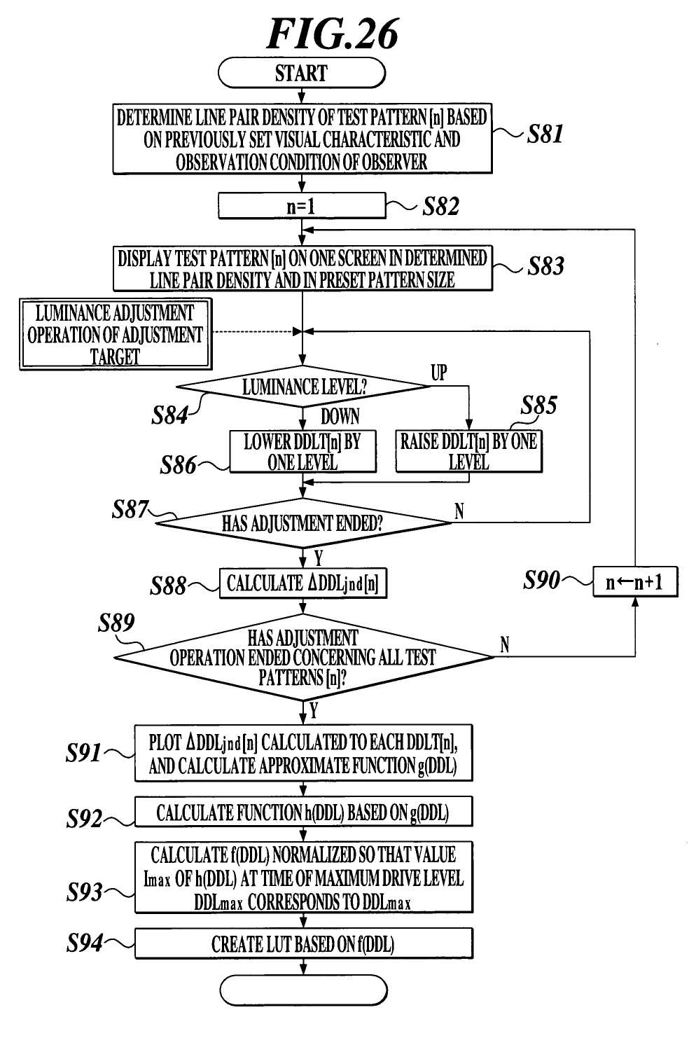

FIG. 26 is a flowchart illustrating the calibration processing. The processing is started when the calibration is selected in an environment setting menu.

In the calibration processing shown in FIG. 26, the information on the observation condition and the visual characteristic of the observer is first read from the storage unit 16, and a line pair density J at the time of displaying each test pattern [n] based on the read information is determined (Step S81).

The determination method of the line pair density is described.

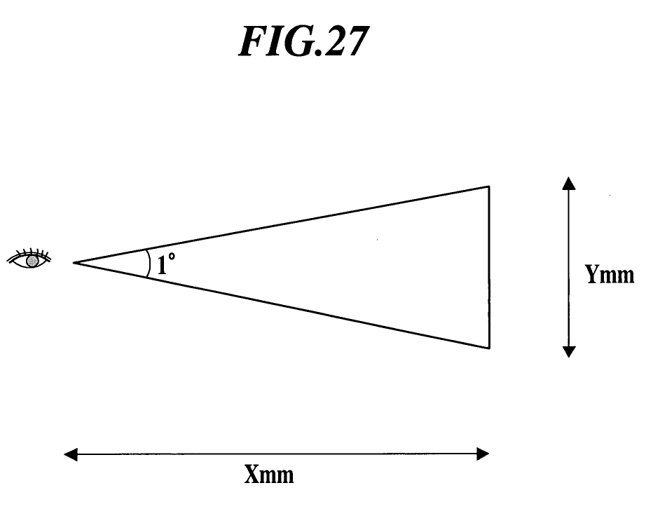

As shown in FIG. 27, when it is supposed that a line pair number to which the visual recognition rate per, view angle is highest (hereinafter the unit of the line pair number is denoted by lp) is C (unit: lp/deg), the observation distance is X (unit: mm), and the visual field range at the one degree of view angle at the time of observation distance X is Y (mm), then the line pair density J (lp/mm) which is the easiest line pair density to recognize can be obtained from the following formula 5.

$$J = \frac{C}{Y} = \frac{C}{2 \cdot X \cdot tan ( \frac{1}{2} )} \qquad formula 5$$

In the case of the test pattern of a line pair, it is known that people's visual recognition rate is the highest at the time of three to five line pairs per visual angle (hereinafter referred to as deg). In the present embodiment, the line pair density J is calculated by applying C=4 (lp/deg). As the observation distance X, a parameter set in the storage unit 16 in advance, i.e. X=500 mm, is applied, and then the line pair density J is calculated as J=0.46 lp/mm from the above formula 5.

Incidentally, although the observation distance is beforehand set as 500 mm and the line pair density J which has the highest recognition rate when the observation distance is 500 mm is set in the present embodiment, the line pair density J is not limited to that. The line pair density J may be calculated in the control unit 11 based on the observation distance which is inputted each time by an operator at the time of performing calibration and the information of the line pair number at which the visual recognition rate per view angle is the highest.

Next, the pattern number n of a test pattern of a display object is set to n=1, which is an initial value (Step S82). When the pattern number n has been set, the data of the test pattern [n] having the pattern number n is read from the storage unit 16, and the data processing is performed to form line pairs at the determined line pair density J. Then, the test pattern [n], in which the line pairs are formed at the determined line pair density, is displayed on the display unit 13 at the preset pattern size of 17.5 mm×17.5 mm (Step S83).

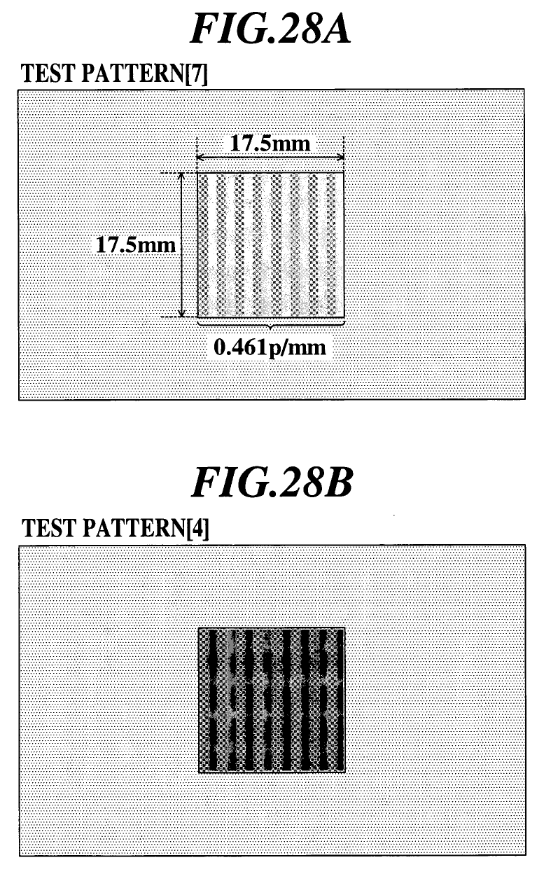

FIG. 28A shows a display example of a test pattern [7].

The control unit 11 performs data processing in order that only one test pattern [7] may be displayed on one screen as shown in FIG. 28A, and that the pattern size of the test pattern [7] may be 17.5 mm×17.5 mm and the line pair density in the pattern may be 0.46 lp/mm, namely in order that the line pairs of 17.5 mm×0.46 lp/mm=8.05 lp may be formed in the pattern of the above-mentioned size. Incidentally, the pattern size is not limited to 17.5 mm×17.5 mm, but the pattern size can be set to other sizes.

Moreover, the background region of the test pattern [n] is driven to be displayed by a drive level DDL_{0.2} corresponding to 20% of luminance level of the maximum luminance level so that the luminance level of the background region becomes one corresponding to 20% of the maximum luminance level in accordance with DICOM standard. The drive level of the background region is common to all the test patterns [n], and all the luminance levels of the background regions of the respective test patterns [n] become the same. For example, when the test pattern [7] is changed and a test pattern [4] is displayed, as shown in FIG. 28B, the luminance level of the background region of the test pattern [5] becomes the same as the luminance level of the background region of the test pattern [7] shown in FIG. 28A.

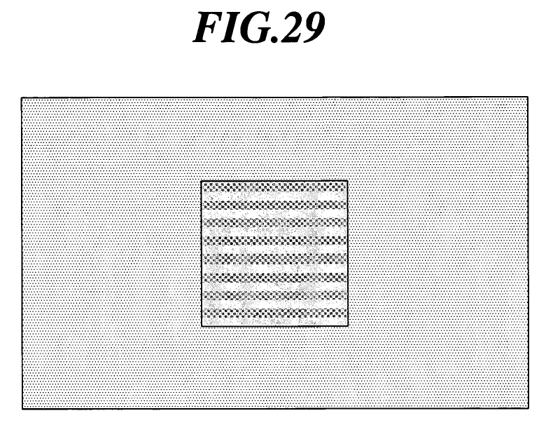

Incidentally, the test pattern [n] may be located to be horizontal lines as shown in FIG. 29.

In such a way, when a test pattern [n] has been displayed, the operator performs an adjustment operation of raising or lowering the luminance level of an adjustment target until the luminance level difference between the adjustment target and the reference target in the test pattern [n] becomes the minimum luminance level difference which can be visually recognized by visual observation through the operation unit 12. The minimum luminance level difference which can be visually recognized means a luminance level difference at the limit of visually recognizing the luminance level difference as a result of an operator's adjusting the luminance level of the adjustment target in accordance with the luminance level of the reference target. To put it concretely, the luminance level is adjusted until a stage at which the operator would be unable to identify the luminance level difference between the adjustment target and the reference target if the luminance level of the adjustment target were lowered by more one level.

In the medical image display apparatus 10, when an operation of luminance adjustment of an adjustment target is made through the operation unit 12, the signal analysis of the operation signal is performed, and it is distinguished which the operation instruction is directed to raising the luminous level or to lowering the luminous level (Step S84). When the operation instruction is directed to raising the luminance level (Step S84; up), the drive level DDLT [n] of the adjustment target is increased by one level, and the adjustment target during being displayed is driven to be displayed at the increased drive level DDLT (Step S85). On the other hand, when the operation instruction is performed in order to lower the luminance (Step S84; down), the drive level DDLT [n] is lowered by one level, and the adjustment target during being displayed is driven to be displayed at the lowered drive level DDLT [n] (Step S86). That is, in response to one operation, a display is performed at a luminance level raised or lowered by one drive level. Incidentally, even when the operation of lowering the luminance level of the adjustment target is repeated, the adjustment operation is made to be restricted lest the luminance level of the adjustment target should be equal to the luminance level of the reference target or less by invalidating the adjustment operation of making the luminance level equal to the drive level of the reference target or less, or by a similar measure.

When the adjustment operation of the drive level has been performed, it is judged whether the adjustment operation has ended or not (Step S87). When the adjustment operation is still being performed through the operation unit 12 (Step S87; N), the processing returns to the process of Step S84, and the luminance level is adjusted in conformity with the inputted operation instruction. On the other hand, when the adjustment operation has ended (Step S87; Y), a drive level DDLT′ [n] of the adjustment target of the test pattern [n] at the time of the adjustment end is judged, and a difference ΔDDL_{jnd} [n] between the drive level DDLT′ [n] and the drive level DDLK [n] of the reference target is calculated (Step S88). The difference ΔDDL_{jnd} [n] corresponds to the minimum luminance level difference which the operator can visually recognize in the test pattern [n].

Subsequently, it is judged whether the adjustment operation mentioned above has ended about all test patterns [n] or not (Step S89). When the adjustment operation has not ended about all of the test patterns [n] (Step S89; N), the pattern number n is incremented by only one (Step S90). Then, the processing returns to the process of Step S83, and the adjustment operation of the next test pattern [n] is repeated. That is, only one test pattern [n] is displayed on one screen, and each test pattern [n] is sequentially changed to be displayed in the order of the pattern number.

Then, when all the test patterns [n] have been displayed and the adjustment operation has ended (Step S89; Y), as shown in FIG. 9, the difference ΔDDL_{jnd} [n] calculated to the drive level DDLK [n] of the reference target of each test pattern [n] is plotted, and an approximate function g(DDL) of each plotted point is calculated (Step S91). That is, each difference ΔDDL_{jnd} [n], which is a discrete value, is interpolated, and a continuous value corresponding to the drive level DDL of all level ranges is obtained. That is, the approximate function g(DDL) is a function showing a correspondence relation between the drive level DDL and the minimum drive level difference ΔDDL_{jnd} [n].

Subsequently, a function h(DDL) of the integral of the calculated function g(DDL) to every drive level DDL is calculated by the formula 3 (Step S92). The above function g(DDL) expresses the inclination of the function h(DDL).

FIG. 10 is a view showing the function h(DDL). The X-axis thereof expresses the drive level DDL, and the Y-axis thereof expresses the function h(DDL) to the drive level DDL. In this case, supposing that a value of the function h(DDL) corresponding to the maximum drive level DDLₘₐₓ which can be driven by the medical image display apparatus 10 is set to Iₘₐₓ, the function h(DDL) (i.e. the values of the Y-axis) is normalized so that the value Imax corresponds to the maximum drive level DDLₘₐₓ, and a function f(DDL) expressing the normalized function h(DDL) is calculated by the formula 4 (Step S93).

Then, a curve shown in FIG. 11 is created. The curve is obtained by substituting the drive levels DDL, which are the units of the X-axis of FIG. 10, with the input levels of image values (Pₘₐₓ in the figure indicates the maximum gradation which can be expressed by the medical image display apparatus 10), and by substituting the function h(DDL), which is the unit of the Y-axis, with the calculated function f(DDL). With this curve, a drive level which realizes a corresponding luminance level from the input level of a pixel value can be obtained so that the input level of the pixel value may be finally displayed at a luminance level visually linear to the input level of the pixel value. That is, the curve is a correction curve for correcting the display gradation characteristic according to the display characteristic of the medical image display apparatus 10 (that is, for correcting a relation between the input value of a pixel value and a luminance level at the time of being actually displayed according to the input level). In the control unit 31, the output values to the input values of this correction curve are calculated and are tabled to create a LUT (Step S94). When the created LUT is stored in the storage unit 16 as the LUT for correcting the display gradation characteristic of the medical image display apparatus 10, the present processing ends.

When a medical image is displayed, the created LUT is referred to, and the drive level (output value) corresponding to the input level (input value) of the pixel value of the medical image is obtained to perform a display and a drive at the drive level. Because the drive level obtained from the LUT is a drive level corrected in order that the luminance level at the time of being driven to be displayed may be in a visually linear relation with the input level of the pixel value, the drive and the display mean that the display gradation characteristic of the medical image display apparatus 10 is corrected according to the display characteristic of the medical image display apparatus 10, and the visual characteristic of the operator.

As mentioned above, according to the fourth embodiment, when the test patterns for calibration are displayed, the test patterns are changed to be displayed so that one pattern is displayed on one screen. Consequently, a fall of the accuracy of the visibility owing to the entering of a test pattern other than the test pattern to be visually recognized into the visual field of an operator can be prevented. Consequently, more accurate calibration can be performed.

Moreover, because the line pair density J is determined as the display condition of the test pattern [n] so that the visual recognition rate becomes the best based on the visual characteristic and the observation condition of the observer to display each test pattern [n] under the determined display condition, the visual recognition rate to the test pattern [n] is improved, and more accurate calibration can be performed.

Furthermore, even when the test pattern [n] to be visually recognized is changed to be displayed, the drive level of the background region of each test pattern [n] is unified to be set to the drive level DDL_{0.2} corresponding to the luminance level of 20% of the maximum luminance level in order that the background level may be displayed at a fixed luminance level (the luminance level of 20% of the maximum luminance level) in conformity with the DICOM standard. Consequently, the influence of the background region to the test pattern [n] can be reduced, and a stable calibration can be performed.

Incidentally, the contents of the description in the present embodiment are suitable examples of the medical image display apparatus 10 to which the present invention is applied, and the present invention is not limited to the contents.

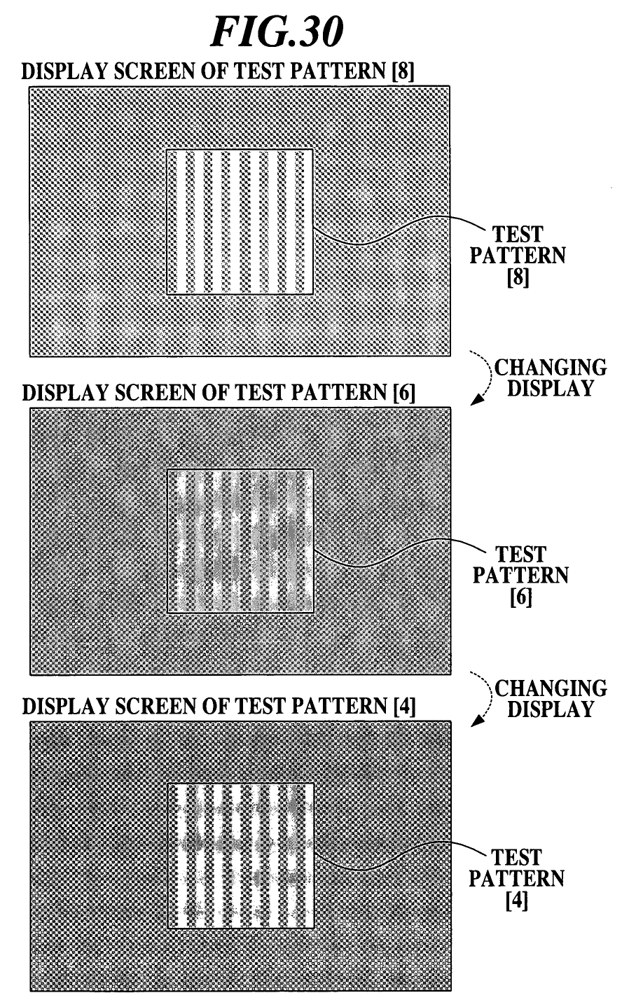

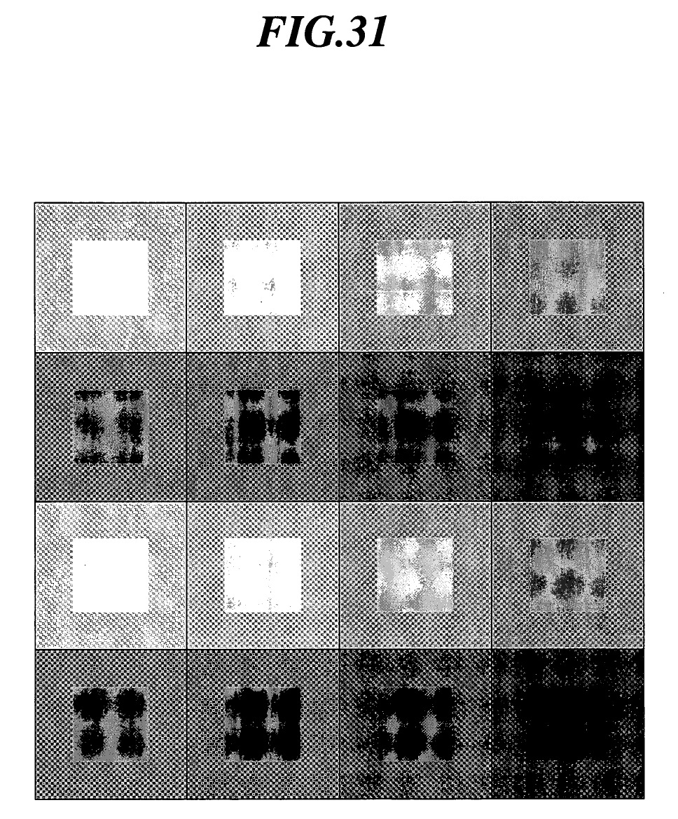

For example, in the descriptions mentioned above, although the background region of any of the test patterns [n] is driven to be displayed at the same drive level (the drive level DDL_{0.2} corresponding to 20% of the maximum luminance level) when each test pattern [n] is changed to be displayed every screen, the present invention is not limited to such a way, but the background region may be varied according to the drive level (luminance level) set to each test pattern [n]. For example, as shown in FIG. 30, the luminance level of the background region of each test pattern [n] is displayed so that the luminance level of the background region becomes the same luminance level as that of the test pattern [n] as follows. That is, when a test pattern [8] is displayed, the background region thereof is driven to be displayed at the same drive level as the drive level DDLK [8] of the reference target thereof; when a test pattern [6] is displayed, the background region thereof is driven to be displayed at the same drive level as the drive level DDLK [6] of the reference target thereof; and when a test pattern [4] is driven to be displayed, the background region thereof is driven to be displayed at the same drive level as the drive level DDLK [4] of the reference target thereof.

By displaying the background region at the luminance level according to each test pattern [n] like that, the background region having the luminance level near the luminance level of the test pattern [n] enters a visual field together with the test pattern [n]. Consequently, the eyes of an operator adapt themselves to the luminance, and the accuracy of visibility becomes good. Consequently, it becomes possible to perform a more accurate calibration. Incidentally, the above-mentioned descriptions are examples. As long as the display gradation of the background region vary according to the test pattern [n], the drive level of the background region may be set to be other values such as an averaged value of the drive levels DDLT [n] and DDLK [n].

All the disclosed contents of Japanese Patent Applications Nos. 2004-176983, 2004-177008, 2004-177073 and 2004-177104, all filed on Jun. 15, 2004, are incorporated in the present application.

1. A display method of a test pattern, comprising making a display section of a medical image display apparatus display one of a plurality of test patterns for correcting a display gradation characteristic of the medical image display apparatus on one screen in a way of changing the test pattern every screen so that the test pattern displayed on each changed screen is displayed at a predetermined ratio to a size of a display screen and a luminance level of a background region of the test pattern is displayed at a different luminance level every change of the screen.

2. The display method of claim 1, wherein
the luminance level of the background region of the test pattern is made to correspond to the luminance level of the test pattern displayed on each changed screen.

3. The display method of claim 1, wherein
the test pattern displayed on each changed screen is displayed at a ratio of 10% of a size of the display screen.

4. A display method of a test pattern, comprising making a display section of a medical image display apparatus display one of a plurality of test patterns for correcting a display gradation characteristic of the medical image display apparatus on one screen on the display section in a way of changing the test pattern every screen so as to vary a display form of the test pattern displayed on each changed screen.

5. The display method of claim 4, wherein
the display form of the test pattern is varied every change of the screen.

6. The display method of claim 4, wherein
the display form of the test pattern is varied in each changed screen.

7. The display method of claim 4, wherein
the display form is a display position of the test pattern.

8. The display method of claim 4, wherein
the display form is a display size of the test pattern.

9. The display method of claim 4, wherein
the display form is a display shape of the test pattern.

10. The display method of claim 4, wherein
a luminance level of a background region of a test pattern displayed on each changed screen is made to correspond to a luminance level of the test pattern.

11. A display method of a test pattern, comprising making a display section of a medical image display apparatus display one of a plurality of test patterns for correcting a display gradation characteristic of the medical image display apparatus on one screen on the display section in a way of changing the test pattern every screen so that the test pattern displayed on each changed screen comprises two display regions to display one display region having a larger display area between them at a lower luminance level than that of another display region.

12. The display method of claim 11, wherein the test pattern displayed on each changed screen is displayed on the whole screen.

13. A display method of a test pattern, comprising making a display section of a medical image display apparatus display one of a plurality of test patterns for correcting a display gradation characteristic of the medical image display apparatus on one screen in a way of changing the test pattern every screen so that a display condition of the test pattern displayed on the screen is determined based on preset information of a visual characteristic and an observation condition of an observer to display the test pattern under the determined display condition.

14. The display method of claim 13, wherein
a background region of the test pattern displayed on each changed screen is displayed at a fixed luminance level.

15. The display method of claim 13, wherein
a background region of the test pattern displayed on each changed screen is made to be displayed at a luminance level corresponding to a luminance level of the test pattern on each changed screen.

16. The display method of claim 13, wherein
each of the test patterns comprises a plurality of line pairs, each of the line pairs comprising two line-like display regions to be displayed at luminance levels different from each other, and
when each of the test patterns is displayed on each changed screen, a density of the line pairs formed in the test pattern is determined based on information of a visual characteristic and an observation condition of an observer as a display condition of the test pattern to display the test pattern at the determined line pair density.

17. The display method of claim 13, wherein
the observation condition is an observation distance from the observer to a display screen and a pattern size of the test pattern.

18. A medical image display apparatus comprising:
a display section for displaying a medical image; and
a control section for making the display section display one of a plurality of test patterns for correcting a display gradation characteristic of the medical image display apparatus on one screen in a way of changing the test pattern every screen so that the test pattern displayed on each changed screen is displayed at a predetermined ratio to a size of a display screen and a luminance level of a background region of the test pattern is displayed at a different luminance level every change of the screen.

19. A medical image display apparatus comprising:
a display section for displaying a medical image; and
a control section for making the display section display one of a plurality of test patterns for correcting a display gradation characteristic of the medical image display apparatus on one screen in a way of changing the test pattern every screen so as to vary a display form of the test pattern displayed on each changed screen.

20. A medical image display apparatus, comprising:
a display section for displaying a medical image; and
a control section for making the display section display one of a plurality of test patterns for correcting a display gradation characteristic of the medical image display apparatus on one screen in a way of changing the test pattern every screen so that the test pattern displayed on each changed screen comprises two display regions to display one display region having a larger display area between them at a lower luminance level than that of another display region.

21. A medical image display apparatus, comprising:
a display section for displaying a medical image; and
a control section for making the display section display one of a plurality of test patterns for correcting a display gradation characteristic in the display section on one screen in a way of changing the test pattern every screen so that a display condition of the test pattern displayed on the screen is determined based on preset information of a visual characteristic and an observation condition of an observer to display the test pattern under the determined display condition.
